(19) United States
(12) Patent Application Publication
(10) Pub. No.: US 2024/0116863 A1
(43) Pub. Date: Apr. 11, 2024

(54) DIPHENYL THIOUREA AND DIPHENYL UREA DERIVATIVES AS INHIBITORS OF ENDOCYTOSIS

(71) Applicants: Indian Institute of Science Education and Research, Mohali (IISER Mohali), Nagar Mohali (IN); Indian Institute of Technology Ropar (IIT Ropar), Rupnagar (IN); Council of Scientific and Industrial Research (CSIR), New Delhi (IN)

(72) Inventors: NIRMAL KUMAR, S.A.S. Nagar Mohali (IN); INDRANIL BANERJEE, S.A.S. Nagar Mohali (IN); IRSHAD MAAJID TAILY, Rupnagar (IN); PRABAL BANERJEE, Rupnagar (IN); CHARANDEEP SINGH, Chandigarh (IN); ANSHUL SHARMA, Chandigarh (IN); RAJESH P. RINGE, Chandigarh (IN); KRISHAN GOPAL THAKUR, Chandigarh (IN); PRIYANKA SINGH, Rupnagar (IN)

(21) Appl. No.: 17/932,386

(22) Filed: Sep. 15, 2022

(51) Int. Cl. C07C 335/12; C07C 275/30

(52) U.S. Cl. CPC C07C 335/12; C07C 275/30

(57) ABSTRACT

The present invention relates to method of inhibition of cellular endocytosis by treating the cells with compound of formula-1 Diphenylthio urea and Diphenylurea derivatives. Said invention also discloses method of treatment of infectious diseases, neurological disease, cancer, kidney disease in a subject in need thereof by administering therapeutically effective concentration of compounds of Formula-1, Diphenylthio urea and Diphenylurea derivatives which are effective inhibitors of cellular endocytosis. Said invention also discloses compounds of Formula-1, Diphenylthio urea and Diphenylurea derivatives and pharmaceutical composition comprising compounds of Formula-1, Diphenylthio urea and Diphenylurea derivatives along with suitable excipients.

### TECHNICAL FIELD

The present invention relates to method of inhibition of cellular endocytosis and treatment of infectious diseases, neurological disease, cancer, kidney disease in a subject in need thereof by administering therapeutically effective concentration of compounds of Formula-1, Diphenylthio urea and Diphenylurea derivatives which are effective inhibitors of cellular endocytosis.

### BACKGROUND OF THE INVENTION

Endocytosis is a form of active cellular transport process, in which substances are brought into the cell from the external environment. The material to be internalized is surrounded by an area of plasma membrane of cell, which then buds off inside the cell to form a vesicle containing the ingested material. There are three primary types of endocytosis: phagocytosis otherwise called as cell eating, pinocytosis otherwise called as cell drinking, and receptor-mediated endocytosis such as clathrin mediated endocytosis. Endocytosis plays a critical role in many disease mechanisms.

Temporary inhibition of endocytosis with small molecules has been proposed for several clinical applications in the treatment of pathogenic infections by viruses, bacteria and fungi, neurological diseases, cancer, and kidney disease. Moreover, a recent study showed that anti-tumour activity of anti-EGFR monoclonal antibodies is augmented in endocytosis-inhibited cells, increasing target receptor availability for antibody binding demonstrating that the endocytosis inhibitors could be used in combination with immunotherapeutic agents to enhance an immune response to a cancer. Enhanced antibody binding to the target receptor increases the efficacy of antibody-dependent cellular cytotoxicity (ADCC) via receptor clustering and retention at the cell surface, leading to improved clinical responses. In addition to the potential application of endocytosis inhibitors as prophylactic and therapeutic agents against pathogenic infections and in the treatment of other diseases and improving clinical responses, endocytosis inhibitors are extensively used as reagents in biomedical research.

Notwithstanding above, employment of endocytosis inhibitors for therapeutic or prophylactic purposes are limited due to their cytotoxic nature. Particularly, high concentration of endocytosis inhibitors is required for the efficient blockage of endocytosis which results in cytotoxicity.

There is a need for the identification of compounds which could effectively block endocytosis at a concentration low enough to prevent cytotoxicity, which could be employed to treat a broad spectrum of diseases including infectious diseases, neurological diseases, cancer or kidney disease where in endocytosis of pathogenetic particles or ligand receptor complexes have been recognized as major contributors in the pathophysiology of said diseases.

The present invention discloses method of inhibition of cellular endocytosis in cell with the compound of formula I. The invention discloses treatment of infectious diseases, neurological disease, cancer, or a kidney disease in a subject in need thereof by administering therapeutically effective concentration of compounds of Formula-1. Compounds of Formula-1 act as efficient inhibitors of endocytosis, which effectively block endocytosis of different ligands (transferrin, epidermal growth factor, cholera toxin B) at lower concentrations than the effective concentrations of the commercially available compounds, without exhibiting cytotoxicity. Moreover, compounds of Formula-1 could also be effectively used in the treatment of pathogenic infections by blocking endocytosis of pathogens, thus employable as broad-spectrum drugs against infectious diseases, as exemplified in inhibiting viral infections such as those of influenza A virus and SARS-CoV-2, which require temporary inhibition of endocytic processes to block virus entry and a consequent inhibition of infection.

### SUMMARY OF THE INVENTION

The present invention relates to a method for inhibition of cellular endocytosis by treatment of cells with the compounds of Formula-I.

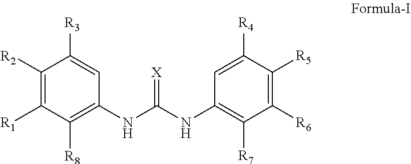

Wherein
X=S or O;
R1=H, Halogen, Alkoxy or Haloalkyl;
R2=H, Halogen, Haloalkyl, Haloalkoxyl, Alkoxyl, Alkyl, Nitro or Benzyl;
R3=H, Halogen, Alkyl or Haloalkyl;
R4=H, Halogen, Alkyl or Haloalkyl,
R5=H, Haloalkyl, Haloalkoxyl, Halogen, Alkoxyl, Alkyl, Nitro or Benzyl;
R6=H, Halogen, Alkoxy or Haloalkyl;
R7=H, Haloalkyl, Alkyl or Halogen;
R8=H, Haloalkyl, Alkyl or Halogen;

Another aspect of the present invention pertains to inhibition of endocytosis of Transferrin, EGF (Epidermal growth factor) and/or cholera toxin in the cell, by treating the cells with therapeutically effective amounts of compounds of Formula-I.

Yet another aspect of the present invention is to provide a method of treatment of infectious diseases, neurological disease, cancer, or a kidney disease in a subject in need thereof by administering to the subject a therapeutically effective amount of a compounds of formula-I.

Yet another aspect of the present invention is to provide a method of treatment of infectious diseases particularly viral infectious diseases specifically by inhibiting the entry of viruses into host cells, in other words by inhibiting endocytosis of viruses into cells, by administering to the subject, therapeutically effective amount of compounds of formula-I. More particularly, to provide a method of treatment for a broad spectrum of viral infectious diseases comprising, but not limited to diseases caused by highly contagious pathogens such as different strains of influenza A virus (Strains: A/X-31/H3N2, A/WSN/33/H1N1, A/Udorn/72/H3N2, and A/NYMC-X311/H3N2) and severe acute respiratory syndrome coronavirus 2 (SARS-CoV-2) (Strains: D614G, Delta (B.1.617.2) and Omicron (B.1.1.529)), by administering therapeutically effective amounts of compounds of Formula-I.

The above and yet other objects and advantages of the present invention will become apparent from the hereinafter set forth detailed description of the Invention as set forth herein.

### BRIEF DESCRIPTION OF THE DRAWINGS

The accompanying drawings illustrate some of the embodiments of the present invention and together with the descriptions, explain the invention. These drawings have been provided by way of illustration and not by way of limitation. The components in the drawings are not necessarily drawn to scale, emphasis instead being placed upon clearly illustrating the principles of the aspects of the embodiments.

FIG. 1A & FIG. 1B: Graphical representation of efficacy comparison between chlorpromazine (commercially available endocytosis inhibitor) and diphenyl thiourea (DPTUD) and diphenyl urea derivatives (DPUD-1, -2, -16, -20, -23) at a concentration of 10 μM (FIG. 1A) and 25 μM (FIG. 1B) in blocking transferrin endocytosis. All data are represented as mean±SD. The P-value was determined using one-way ANOVA with multiple comparisons w.r.t. chlorpromazine. ns: P>0.05, *P<0.05, **P<0.01, ***P<0.001, ****P<0.0001.

FIG. 2A & FIG. 2B: Graphical representation of efficacy comparison between chlorpromazine and methyl-β-cyclodextrin (commercially available endocytosis inhibitors) and diphenyl thiourea (DPTUD) and diphenyl urea derivatives (DPUD-1, -2, -16, -20, -23) at a concentration of 10 μM in blocking EGF (FIG. 2A) and cholera toxin B (FIG. 2B) endocytosis. All data are represented as mean±SD. The P-value was determined using one-way ANOVA with multiple comparisons w.r.t. chlorpromazine. ns: P>0.05, *P<0.05, **P<0.01, ***P<0.001, ****P<0.0001.

FIG. 3: Cytotoxicity comparison between chlorpromazine (commercially available endocytosis inhibitor) and diphenyl thiourea (DPTUD) and diphenyl urea derivatives (DPUD-1, -2, -16, -20, -23) as indicated by the number of viable cells upon 12 h treatment with the compounds (50 μM). All data are represented as mean±SD. The P-value was determined using one-way ANOVA with multiple comparisons w.r.t. DMSO. ns: P>0.05, *P<0.05, **P<0.01, ***P<0.001, ****P<0.0001.

FIG. 4A & FIG. 4B: Effect of diphenyl thiourea (DPTUD) and diphenyl urea derivatives (DPUD-1, -2, -16, -20, -23) in blocking cellular entry (FIG. 4A) and infection (FIG. 4B) of influenza A virus (A/X-31/H3N2 strain). All data are represented as mean±SD, The P-value was determined using one-way ANOVA with multiple comparisons w.r.t. DMSO. ns: P>0.05, *P<0,05, **P<0.01, ***P<0.001, ****P<0.0001.

FIG. 5A & FIG. 5B: Effect of diphenyl thiourea (DPTUD) and diphenyl urea derivatives (DPUD-1, -2, -16, -20, -23) in blocking cellular entry of pseudotyped SARS-CoV-2 (FIG. 5A) and infection of SARS-CoV-2 (D614G strain) (FIG. 5B). All data are represented as mean±SD. The P-value was determined using one-way ANOVA with multiple comparisons w.r.t. DMSO. ns: P>0.05, *P<0.05, **P<0.01, ***P<0.001, ****P<0.0001.

FIG. 6A: Heatmaps showing results of high-content infection screens with IAV X-31 (H3N2), WSN (H1N1) and SARS-CoV-2 (D614G) in A549 and Vero-E6 cells, respectively, for 1,3-diphenylurea (DPU) and 23 DPU derivatives (DPUDs).

FIG. 6B: Molecular structures of DPU and the ‘hit’ compounds (DPUD-1, -2, -16, -20 and -23).

FIG. 6C: High-content microscopy images of IAV- and SARS-CoV-2-infected cells, treated with DMSO, DPU, DPU-1 and BafA1/niclosamide. Nuclei were stained with Hoechst (magenta), and the viral NP/N proteins (green) were detected by IIF. Scare bars, 50 μm.

FIG. 6D: Reduced infection by DPUD-1, -2, -16, -20 and -23 in A549 cells infected with IAV Udorn (H3N2) and NYMC (H3N2) strains. n=3 biologically independent experiments. All data are represented as mean+SD. The P-value was determined using one-way ANOVA with multiple comparisons w.r.t. DMSO. ns: P>0.05, *P<0.05, **P<0.01, ***P<0.001, ****P<0.0001.

FIG. 6E: Reduced infection by DPUD-1, -2, -16, -20 and -23 (10 μM) in Vero-E6 cells infected with SARS-CoV-2 Delta (B.1.617.2) and Omicron (B.1,1.529) strains. n=3 biologically independent experiments. All data are represented as mean±SD. The P-value was determined using one-way ANOVA with multiple comparisons w.r.t. DMSO. ns: P>0.5, *P<0.05, **P<0.01, ***P<0.001, ****P<0.0001.

FIG. 7A: Schematic representation of the experimental design of in vivo efficacy assessment of DPUDs against IAV infection.

FIG. 7B: Graph showing % body weights of the uninfected mice, and IAV-infected mice that were treated with DPUD-1, Oseltamivir acid, and DMSO.

FIG. 7C: Survival of the IAV-infected mice up to 21 days post-infection that were treated with DPUD-1, Oseltamivir acid, and DMSO.

FIG. 7D: RT-PCR of IAV (WSN) NP gene from the lungs of infected mice (n=3) treated with DMSO, DPUD-1, Oseltamivir acid, sacrificed on day 6 post-infection. All data are represented as mean±SD. The P-value was determined using one-way ANOVA with multiple comparisons w.r.t, DMSO. ns: P>0.05, *P<0.05, **P<0.01, ***P<0.001, ****P<0.0001.

FIG. 7E: Virus titres from the lungs of infected mice (n=3) treated with DMSO, DPUD-1, Oseltamivir acid, sacrificed on day 6 post-infection. All data are represented as mean±SD. The P-value was determined using one-way ANOVA with multiple comparisons w.r.t. DMSO. ns: P>0.05, *P<0.05, **P<0.01, ***P<0.0001, ****P<0.0001.

FIG. 7F: Images and results of virus plaque assays in MDCK cells from the supernatants of A549 cells infected with IAV (WSN) treated with DMSO,DPUD-1, Oseltamivir acid. All data are represented as mean±SD. The P-value was determined using one-way ANOVA with multiple comparisons w.r.t. DMSO. ns: P>0.05, *P<0.05, **P<0.01, ***P<0.001, ****P<0.0001.

FIG. 7G: Brightfield images and results of virus plaque assays from serial viral passage experiments. All data are represented as mean+SD. The P-value was determined using one-way ANOVA with multiple comparisons w.r.t, DMSO. ns: P>0.05, *P<0.05, **P<0.01, ***P<0.001, ****P<0.0001.

FIG. 8A: Schematic diagram showing the multi-step IAV entry process.

FIG. 8B: Binding assay.

FIG. 8C: Endocytosis assay.

FIG. 8D: HA acidification assay.

FIG. 8E: Uncoating assay.

FIG. 8F: vRNP nuclear import assay.

FIG. 8G: Schematic diagram showing the experimental design of PM bypass infection assay.

FIG. 8H: Plasma membrane (PM) bypass infection assay in cells treated with DPUDs (10 μM) till fusion.

FIG. 8I: PM bypass infection assay in cells in which, DPUDs (10 μM) were added 1 h post-fusion and kept on for the rest 9 h.

FIG. 8J: Images from live cell microscopy, monitoring influenza A virus entry. Influenza A viruses were fluorescently labeled with the lipophilic dye SP-DiOC18(3) (green) and internalized in A549 cells expressing the early endosomal marker Rab5-RFP (red) in presence of DMSO or DPUD-1.

FIG. 9A: Confocal images of A549 cells treated with DPUD-1 (10 μM) or DMSO for 1 h. Antibodies were used to stain EEA1 (cyan) and LAMP1 (red). Phalloidin-AF647 and Hoechst were used to stain actin filaments (yellow) and nuclei (magenta), respectively.

FIG. 9B: Quantification of EEA1 fluorescence intensities in A549 cells treated with DPUDs (10 μM) for 1 h. All data are represented as mean±SD. The P-value was determined using one-way ANOVA with multiple comparisons w.r.t. DMSO. ns: P>0.05, *P<0.05, **P<0.01, ***P<0.001, ****P <0.0001.

FIG. 9C: Time of DPUD addition assay for IAV (X-31) infection in A549 cells.

FIG. 9D: Time of DPUD addition assay for SARS-CoV-2 (D614G) infection in Vero-E6 cells.

FIG. 10A: The workflow of the high-content infection screens performed in A549 and Vero-E6 cells using IAV (X-31, H3N2 and WSN/33, H1N1) and SARS-CoV-2 (D614G) strains, respectively.

FIG. 10B: High-content microscopy images of IAV- and SARS-CoV-2-infected cells, treated with DMSO or DPU, DPU-1, -2, -16, -20, and -23, or BafA1/niclosamide.

FIG. 10C: Relative expression of SARS-CoV-2 N and E genes, detected by RT-PCR, in the supernatants of cells treated with DMSO or DPU. DPU-1, -2, -16, -20, and -23, or niclosamide.

FIG. 10D: Western blots showing inhibition of IAV infections with the X-31 and WSN strains in A549 cells using anti-HA antibody. GAPDH served as a loading control.

FIG. 10E: Viral plaque assay from the supernatants of A549 cells infected with IAV WSN strain treated with DMSO or DPU, DPU-1, -2, -16, -20, and -23, or niclosamide.

FIG. 11A: Graphs showing concentration-dependent effect of DPUDs against IAV (X-31) infection in A549 and Vero-E6 cells to calculate the half-maximal inhibitory concentration (IC50).

FIG. 11B: Graphs showing concentration-dependent effect of DPUDs against IAV (WSN) infection in A549 and Vero-E6 cells to calculate the half-maximal inhibitory concentration (IC50).

FIG. 11C: Graphs showing concentration-dependent effect of DPUDs against SARS-CoV-2 (D614G) infection in A549 and Vero-E6 cells to calculate the half-maximal inhibitory concentration (IC50).

FIG. 11D: Cytotoxic concentration (CC50) values corresponding to each DPUD compound in A549 cells.

FIG. 11E: Cytotoxic concentration (CC50)values corresponding to each DPUD compound in Vero-E6 cells.

FIG. 11F: Selectivity indices of the compounds for IAV X-31 and WSN, and SARS-CoV-2 (D614G) infection.

FIG. 12A: Graph showing % body weights of the uninfected mice, and IAV-infected mice that were treated with DPUD-2, -16, -20, and -23, Oseltamivir acid, and DMSO.

FIG. 12B: Images of viral plaque assays.

FIG. 13A: High-content images of IAV (X-31) cellular entry assays performed in DMSO,DPU, DPUD-1, DPUD-2,DPUD-16, DPUD-20,DPUD-23, No virus/CPZ/BafA1-treated A549 cells.

FIG. 13B: Graphs showing concentration-dependent effect of DMSO, DPU, DPUDs (DPUD-1, 2, 16, 20, 23), Niclosamide on HIV-based pseudotyped SARS-CoV-2 (D614G) infections in HEK 293T-hACE2 cells.

FIG. 13C: Graphs showing concentration-dependent effect of DMSO, DPU, DPUDs (DPUD-1, 2, 16, 20, 23), Niclosamide on HIV-based pseudotyped SARS-COV-2 (Delta) infections in HEK 293T-hACE2 cells.

FIG. 13D: Graphs showing concentration-dependent effect of DMSO, DPU, DPUDs (DPUD-1, 2, 16, 20, 23), Niclosamide on HIV-based pseudotyped SARS-CoV-2 (Omicron) infections in HEK 293T-hACE2 cells.

FIG. 14: High-content images of AF488-conjugated EGF (green), transferrin (green) and CTXB (green) uptake, and LysoTracker Red DND-99 (green) accumulation in DPTUD and DPUD-treated A549 cells.

FIG. 15A,15B,15C,15D,15E,15F: Confocal images of A549 cells treated with DMSO (FIG. 15A), DPU (FIG. 15B), DPUD-2 (FIG. 15C), DPUD-16 (FIG. 15D), DPUD-20 (FIG. 15E), DPUD-23 (FIG. 15F), for 1 h. Antibodies were used to stain EEA1 (cyan) and LAMP1 (red), Phalloidin-AF647 and Hoechst were used to stain actin filaments (yellow) and nuclei (magenta), respectively.

### DETAILED DESCRIPTION OF THE INVENTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification are to be understood as being modified in all instances by the term “about”.

It is noted that, unless otherwise stated, all percentages given in this specification and appended claims refer to percentages by weight of the total composition. Thus, before describing the present invention in detail, it is to be understood that this invention is not limited to particularly exemplified systems or process parameters that may of course, vary.

It is also to be understood that the terminology used herein is for the purpose of describing embodiments of the invention only and is not intended to limit the scope of the invention in any manner. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control. It must he noted that, as used in this specification and the appended claims, the singular forms “a,” “an” and “the” include plural referents unless the content clearly dictates otherwise. Thus, for example, reference to a “polymer” may include two or more such polymers.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein, the terms “comprising” “including,” “having” “containing,” “involving,” and the like are to be understood to be open-ended, i.e., to mean including but not limited to.

As used herein, the term “therapeutically effective amount,” in the context of treating or preventing a disease or condition (e.g., a cancer) is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individuals to be treated, the formulation of the composition, the assessment of the medical situation, and other relevant factors.

As used herein, the terms “treatment,” “treating,” and the like, refer to administering an agent, to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a viral infection) and/or adverse effect attributable to the disease or condition.

The present invention relates to the provision of method of treatment of an infectious disease, a neurological disease, cancer, or a kidney disease in a subject in need thereof, comprising administering to the subject, a therapeutically effective amount of a compound of formula-I:,

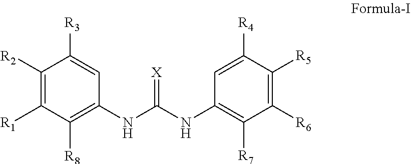

Wherein
X=S or O;
R1=H, Halogen, Alkoxy or Haloalkyl;
R2=H, Halogen, Haloalkyl, Haloalkoxyl, Alkoxyl, Alkyl, Nitro or Benzyl;
R3=H, Halogen, Alkyl or Haloalkyl;
R4=H, Halogen, Alkyl or Haloalkyl,
R5=H, Haloalkyl, Hoiloalkoxyl, Halogen, Alkoxyl, Alkyl, Nitro or Benzyl;
R6=H, Halogen, Alkoxy or Haloalkyl;
R7=H, Haloalkyl, Alkyl or Halogen;
R8=H, Haloalkyl, Alkyl or Halogen;

In a specific embodiment of method of treatment of an infectious disease, a neurological disease, cancer, or a kidney disease, the compound of formula 1 is a compound, wherein X=S; R2, R5, R7, R8=H; R1, R3, R4, R6=Haloalkyl.

In a specific embodiment of method of treatment of an infectious disease, a neurological disease, cancer, or a kidney disease, the compound of formula 1 is a compound, wherein X=O; R2, R5, R7, R8=H; R1, R3, R4, R6=Haloalkyl.

In a specific embodiment of method of treatment of an infectious disease, a neurological disease, cancer, or a kidney disease, the compound of formula 1 is a compound, wherein X=O; R2, R5=Haloalkyl; R1, R3, R4, R6, R7, R8=H.

In a specific embodiment of method of treatment of an infectious disease, a neurological disease, cancer, or a kidney disease, the compound of formula 1 is a compound, wherein X=O; R2, R5, R7, R8=H; R1, R3, R4, R6=Halogen.

In a specific embodiment of method of treatment of an infectious disease, a neurological disease, cancer, or a kidney disease, the compound of formula 1 is a compound, wherein X=O; R2, R5, R7 R8=H; R1, R3, R4, R6=F or Br.

In a specific embodiment of method of treatment of an infectious disease, a neurological disease, cancer, or a kidney disease, the compound of formula 1 is a compound of Formula-I, wherein X=O; R2, R5=Halo alkoxyl; R1 R3, R4, R6, R7, R8=Hydrogen.

In a specific embodiment of method of treatment of an infectious disease, a neurological disease, cancer, or a kidney disease, the compound of formula 1 is 1,3-bis(3,5-bis(trifluoromethyl)phenyl)thiourea, represented as DPTUD.

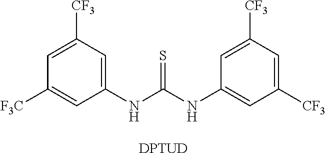

In a specific embodiment of method of treatment of an infectious disease, a neurological disease, cancer, or a kidney disease, the compound of formula 1 is 1,3-bis(3,5-bis(trifluoromethyl)phenyl)urea, represented as DPUD1

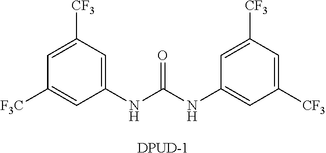

In a specific embodiment of method of treatment of an infectious disease, a neurological disease, cancer, or a kidney disease, the compound of formula 1 is 1,3-bis(4-(trifluoromethyl)phenyl)urea, represented as DPUD2

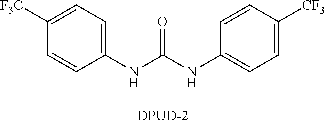

In a specific embodiment of method of treatment of an infectious disease, a neurological disease, cancer, or a kidney disease, the compound of formula 1 is 1,3-bis(3,5-difluorophenyl)urea, represented as DPUD16.

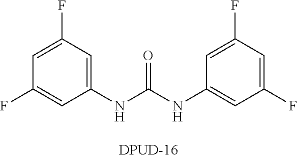

In a specific embodiment of method of treatment of an infectious disease, a neurological disease, cancer, or a kidney disease, the compound of formula 1 is 1,3-bis(4-(trifluoromethophenyl)urea, represented as DPUD20.

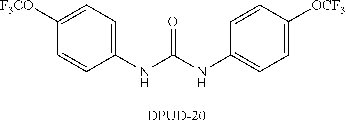

In a specific embodiment of method of treatment of an infectious disease, a neurological disease, cancer, or a kidney disease, the compound of formula 1 is 1,3-bis(3,5-dibromophenyl)urea, represented as DPUD23.

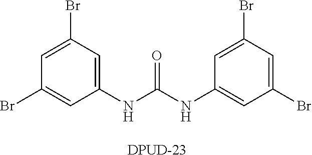

In specific embodiments, the subject comprises Human or Animal and the infectious diseases comprise diseases caused by viral, bacterial, and fungal infections,

In particular embodiments, Viral infections comprise but not limited to diseases caused by Influenza A Virus and Severe acute respiratory syndrome coronavirus 2; Bacterial infections comprise but not limited to infections caused by E. coli, Tuberculosis, Salmonella and fungal infections comprise but not limited to infections caused by Candida, Aspergillus, Cryptococcus.

In a specific embodiment of method of treatment of an infectious disease, a neurological disease, cancer, or a kidney disease, the therapeutically effective amount of the compound of Formula-I ranges from 1 mg-10 mg/kg of body weight.

In a specific embodiment of method of treatment of an infectious disease, a neurological disease, cancer, or a kidney disease, the compound of Formula-I is administered in the form of a pharmaceutical composition, where in the pharmaceutical composition comprises the compound of formula-I and at least one excipient selected from DMSO (Dimethyl sulfoxide).

In a specific embodiment of method of treatment of an infectious disease, a neurological disease, cancer, or a kidney disease, the compound of Formula-I is administered orally, intraperitoneally, intravenously, or intranasally.

Another aspect of the present invention relates to the provision of method of inhibition of endocytosis in a cell, comprising treating the cells with a compound of formula-I:

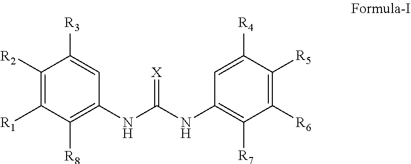

Wherein
X=S or O;
R1=H, Halogen, Alkoxy or Haloalkyl;
R2=H, Halogen, Haloalkyl, Haloalkoxyl, Alkoxyl, Alkyl, Nitro or Benzyl;
R3=H, Halogen, Alkyl or Haloalkyl;
R4=H, Halogen, Alkyl or Haloalkyl,
R5=H, Haloalkyl, Hoiloalkoxyl, Halogen, Alkoxyl, Alkyl, Nitro or Benzyl;
R6=H, Halogen, Alkoxy or Haloalkyl;
R7=H, Haloalkyl, Alkyl or Halogen;
R8=H, Haloalkyl, Alkyl or Halogen;
R8=H, Haloalkyl, Alkyl or Halogen;

In an embodiment of method of inhibition of endocytosis in a cell, the cells are treated with the compound of formula-I at a concentration of 0.1 μM to 25 μM for 10 minutes to 24 hours at a temperature of 37° C.

In an embodiment of method of inhibition of endocytosis in a cell, the compound of formula-I inhibits the endocytosis of endocytic cargoes comprising Transferrin, EGF, and cholera toxin into the cell.

In an embodiment of method of inhibition of endocytosis in a cell, the compound of formula-I inhibits endocytosis of pathogens including viruses, bacteria and fungi into the cell.

In a particular embodiment of method of inhibition of endocytosis in a cell, the compound of formula-I inhibits endocytosis of viruses comprising SARS-CoV-2 or influenza virus into the cell.

In a specific embodiment of method of inhibition of endocytosis in a cell, the compound of formula 1 is 1,3-bis(3,5-bis(trifluoromethyl)phenyl)thiourea, represented as DPTUD.

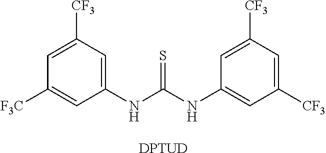

In a specific embodiment of method of inhibition of endocytosis in a cell, the compound of formula 1 is 1,3-bis(3,5-bis(trifluoromethyl)phenyl)urea, represented as DPUDI1

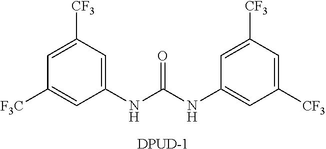

In a specific embodiment of method of inhibition of endocytosis in a cell, the compound of formula 1 is 1,3-bis(4-(trifluoromethyl)phenyl)urea, represented as DPUD2

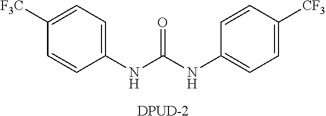

In a specific embodiment of method of inhibition of endocytosis in a cell, the compound of formula 1 is 1,3-bis(3,5-difluorophenyl)urea, represented as DPUD16.

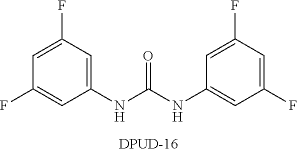

In a specific embodiment of method of inhibition of endocytosis in a cell, the compound of formula 1 is 1,3-bis(4-(trifluoromethoxy)phenyl)urea, represented as DPUD20.

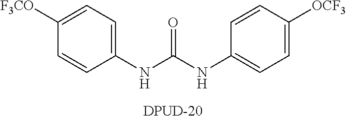

In a specific embodiment of method of inhibition of endocytosis in a cell, the compound of formula 1 is 1,3-bis(3,5-dibromophenyl)urea, represented as DPUD23.

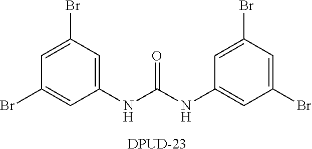

Yet another aspect of the claimed invention pertains to compounds of Formula-I

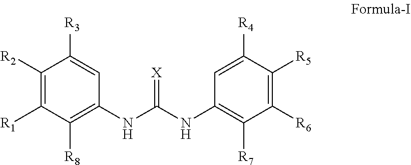

Wherein
X=S or O;
R1=H, Halogen, Alkoxy or Haloalkyl;
R2=H, Halogen, Haloalkyl, Haloalkoxyl, Alkoxyl, Alkyl, Nitro or Benzyl;
R3=H, Halogen, Alkyl or Haloalkyl;
R4=H, Halogen, Alkyl or Haloalkyl,
R5=H, Haloalkyl, Hoiloalkoxyl, Halogen, Alkoxyl, Alkyl, Nitro or Benzyl;
R6=H, Halogen, Alkoxy or Haloalkyl;
R7=H, Haloalkyl, Alkyl or Halogen;
R8=H, Haloalkyl, Alkyl or Halogen;

In a specific embodiment, the compound of Formula-I is a compound wherein X=S; R2, R5, R7, R8=H; R1, R3, R4, R6=Haloalkyl.

In a specific embodiment, the compound of Formula-I is a compound, wherein X=O; R2, R5, R7, R8=H; R1, R3, R4, R6=Haloalkyl.

In a specific embodiment, the compound of Formula-I is a compound, wherein X=O; R2, R5=Haloalkyl; R1, R3, R4, R6, R7, R8=H.

In a specific embodiment, the compound of Form-I is a compound, wherein X=O; R2, R5, R7, R8=R1, R3, R4, R6=Halogen.

In a specific embodiment, the compound of Formula-I is a compound, wherein X=O; R2, R5, R7, R8=H; R1, R3, R4, R6=F or Br.

In a specific embodiment, the compound of Formula-I is a compound, wherein X=O; R2, R5=Halo alkoxyl; R1, R3, R4, R6, R7, R8=Hydrogen.

In a specific embodiment, the compound of formula I is 1,3-bis(3,5-bis(trifluaromethyl)phenyl)thiourea, represented as DPTUD.

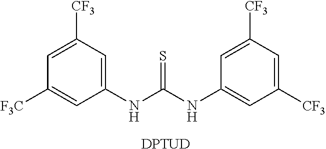

In a specific embodiment, the compound of formula 1 is 1,3-bis(3,5-bis(trifluoromethyl)phenyl)urea, represented as DPUD1

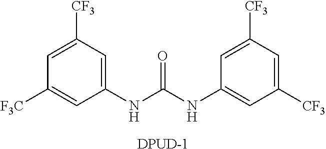

In a specific embodiment, the compound of formula 1 is 1,3-bis(4-(trifluoromethyl)phenyl)urea, represented as DPUD2

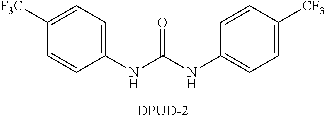

In a specific embodiment, the compound of formula I is 1,3-bis(3,5-difluorophenyl)urea, represented as DPUD16.

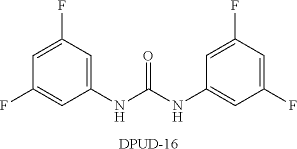

In a specific embodiment, the compound of formula 1 is 1,3-bis(4-(trifluoromethoxy)phenyl)urea, represented as DPUD20.

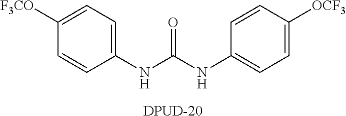

In a specific embodiment, the compound of formula 1 is 1,3-bis(3,5-dibromophenyl)urea, represented as DPUD23.

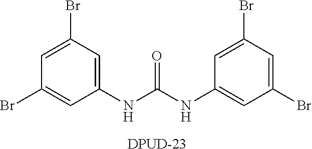

Yet another aspect of the invention pertains to pharmaceutical composition comprising compounds of Formula 1 or a pharmaceutically acceptable salt thereof and at least one pharmaceutically acceptable excipient.

In specific embodiment of the pharmaceutical composition comprising compounds of Formula 1 or a pharmaceutically acceptable salt thereof, the pharmaceutically acceptable excipient comprises DMSO.

Compounds of Formula-1 efficiently blocked the endocytosis at lower concentrations than the effective concentrations of the commercially available compounds employed as endocytosis inhibitors such as Chlorpromazine (CPZ) in clathrin-mediated endocytosis of transferrin and EGF, and methyl-β-cyclodextrin (MβCD) in CTXB endocytosis. Compounds of Formula-1 also blocked the endocytosis in general and were found to be effective in curtailing the pathogenic infections that happen via endocytosis. For instance, compounds of Formula-1 have been demonstrated to effectively block the cellular entry and infection of influenza A virus and BARS-CoV-2 virus. Host cell entry is the first and a critical step in the life cycle of viruses, and this process constitutes a target for therapeutic inhibition. Since many viruses depend on cellular endocytosis to enter the host cell and establish infection, blocking virus endocytosis represents a favourable target for drug development as inhibition of this early phase of infection blocks the subsequent stages of the infection cycle. Endocytosis assay for influenza A virus (IAV) A/X-31/H3N2 strain conducted in cells treated with the diphenyl thiourea and diphenyl urea derivatives demonstrated 68.4-83.4% inhibition in viral entry and >99% inhibition in IAV infection. Similarly treatment with diphenyl thiourea and diphenyl urea derivatives inhibited pseudotyped SARS-CoV-2 cellular entry by about 83.3-99.6% and inhibited infection with the different SARS-CoV-2 strains (D614G, Delta, Omicron) by 97.1-99.5%. The diphenyl thiourea and diphenyl urea derivatives show potent activity at a concentration 10 μM, whereas the inhibitory concentrations of chlorpromazine and methyl-β-cyclodextrin, known inhibitors of endocytosis are 70 μM (7× higher) and 1 mM (100× higher), respectively. Further the diphenyl thiourea and diphenyl urea derivatives did not show cytotoxicity even at 50 μM concentration (5× higher than working concentration). However, chlorpromazine showed very high cytotoxicity (>75%) at the same concentration.

### EXAMPLES

### Synthesis of 1,3-diphenylurea derivatives (DPUDs)

All reactions were carded out in oven-dried glasswares. All solvents and starting materials were obtained from commercial sources. The developed chromatogram was analysed by UV lamp (254 nm) or p-anisaldehyde solution. Products were purified by silica gel (mesh size 230-400) column chromatography. The 1H NMR and 13C NMR spectra were recorded in deuterated chloroform (CDCl3) and deuterated methanol (MeOD) as per requirement. Chemical shifts of 1H and 13C NMR spectra are expressed in parts per million (ppm). All coupling constants are absolute values and are expressed in hertz. The description of the signals includes the following: s=singlet, d=doublet, dd=doublet of doublet, t=triplet, dt=doublet of triplet, q=quartet, dq=doublet of quartet, br=broad, and m=multiple. Purity of the synthesized molecules was determined by HPLC chromatograms using SunFire (C8, 5 μm, 4.6×250 mm) Waters HPLC column with acetonitrile and water as solvents. The HPLC chromatogram of DPUD-1 was obtained by using SunFire (Prep Silica, 5 μm, 4.6×250 mm) Waters HPLC column with isopropylalcohol and n-hexane as solvents.

DPUD-1 synthesis was carried out as follows: The isocyanate was dissolved in tetrahydrofuran (THF), and the corresponding amine was added gravimetrically. After completion of the reaction (monitored by thin-layer chromatography, TLC), THF was removed under vacuum. The crude DPUD-1 was purified by washing with dichloromethane using vacuum filtration, DPUD-2 to DPUD-23 were synthesized as previously described (Wang, M et al. (2018); Bagdasarian, A. L. et al. (2020); Callison, J. et al. (2012); Song, H. X. et al. (2019); Zhao, Y. et al. (2020); Zhou, S. et al. (2013); Park, J. N. et al. (2009); Ricci, A. et al. (2004): 1,3-diphenyl urea (DPU) was procured from commercial source (Merck). To a solution of aromatic amine (1.0 equiv.) in CH₂Cl₂, DABCO (0.1 mmol, 0.1 equiv.) and (Boc)₂O (0.5 equiv.) were successively added. After completion of the reaction as detected by TLC, the reaction mixture was cooled to 0° C. and n-hexane was then added. The resulting solid was collected and further washed with cold water and diethyl ether to afford the corresponding DPUDs.

### Synthesis of Diphenyl Thiourea [DPTUD]

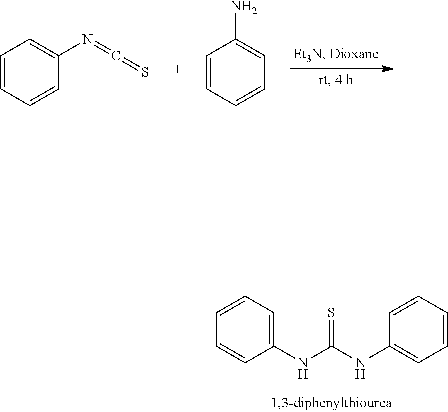 [SMILES: S=C=Nc1ccccc1 ; Nc1ccccc1 ; S=C(Nc1ccccc1)Nc1ccccc1]

To a mixture of 0.3 mol of phenylisothiocyanate in 2.0 mL of 1,4-dioxane, was added 1 mol of triethylamine at room temperature. After stirring for 5 min, the reaction was charged with 0.3 mol aniline and the reaction was continued for approximately 4 hours. The completion of the reaction was monitored by TLC. After the completion, ice-cold water was poured into the reaction mixture with continuous stirring, and solid obtained was filtered and crystallized by appropriate solvent to furnish the desired 1,3-diphenylthiourea.

### Cells and Viruses

A549, MDCK, and HEK293T cells were obtained from American Type Culture Collection (ATCC). HEK 293T-hACE-2 cells were obtained from BEI Resources, NIH (#NR-52511). Vero-E6 cells were acquired from National Centre for Cell Science (NCCS) Pune, India. All cells were maintained in DMEM (Dulbecco's Modified Eagle's Medium, Gibco), supplemented with 10% FBS (Merck), Non-essential amino acids (Invitrogen), and Penicillin Streptomycin Glutamine (Gibco) in 5% CO2 at 37° C. Purified IAV X-31(H3N2) was purchased from Microbiologics (previously Virapur), USA. X-31 virus was propagated in pathogen-free chicken eggs at 33-37° C. for 2 days, following which, the allantoic fluid was harvested. Virus was concentrated by two rounds of 10-40% sucrose gradient high-speed centrifugation, and the purified virus was harvested from the viral bands. The purified virus was resuspended in formulation buffer (40% sucrose, 0.02% BSA, 20 mM HEPES, pH 7.4, 100 mM NaCl and 2 mM MgCl2), and aliquoted and stored in −80° C. until use. The viral titre was determined in MDCK, cells. The median tissue culture infectious dose (TCID50) of purified X-31 virus was determined as 1.95×109. IAV WSN (A/WSN/1933) (H1N1), IAV Udorn (A/Udorn/72) (H3N2) and IAV NYMC_X311 (A/Brisbane/1/2018) (H3N3) strains were kind gifts from Yohei Yamauchi (University of Bristol, UK). IAV WSN, Udorn and NYMC strains were propagated in MDCK cells, concentrated by centrifugation, and stored at −80° C., until use. SARS-CoV-2 viruses were cultured by inoculating Vero-E6 cells with fresh COVID-19 nasal swab sample. Viruses were confirmed to be SARS-CoV-2 D614G, Delta (B.1.617.2) and Omicron (B.1.1.529) strains. All the virus culture work and anti-viral assays were carried out in the BSL-3 laboratory with all the relevant ethical and biological safety clearances by institutional committees. Virus stock was titrated by using quantitative real time PCR (qRT-PCR) and plaque assay and stored at −80° C. until use.

### Antibodies and Reagents

Anti IAV-NP (HB65) and anti-IAV-M1 (HB64) mouse monoclonal antibodies producing hybridoma cell lines were obtained from ATCC. Mouse monoclonal anti-IAV-HA (H3SKE), anti-IAV-HA (A1) specific for post-acid conformation of HA, and rabbit polyclonal anti-IAV-HA (Pinda) were used as previously described (Banerjee. I. et al (2013)). Rabbit anti-SARS-CoV2-N (200-401-A50) was purchased from Rockland Inc. AlexaFluor (AF)-conjugated secondary antibodies, Hoechst 33342, phalloidin-AF647 and Lysotracker-RED DND-99, SP-DiOC18(3) were purchased form Invitrogen. Bafilomycin A1 (BafA1), cycloheximide, chlorpromazine (CPZ), and methylbetacyclodextrin (MβCD) were purchased from Merck. Oseltamivir Acid (MedChemExpress) hydroxychloroquine (HCQ), regorafenib and sorafenib (BLDpharm), niclosamide (Merck) and favipiravir (Merck) were procured from commercial sources. Rabbit polyclonal anti-EEA1(#3288s) and mouse monoclonal anti-Lamp1 (#15665) antibodies were purchased from Cell Signaling Technology.

### High-Content Screening of DPUDs

IAV and SARS-CoV-2 infection assays were performed in IAV infection medium (DMEM with 0.2% BSA and 50 mM HEPES pH 6.8) and SARS-CoV-2 infection medium (DMEM, serum-free), respectively. DPUDs, control inhibitors (BafA1, regorafenib and sorafenib for IAV infection; favipiravir, hydroxychloroquine (HCQ), and niclosamide for SARS-CoV-2 infection), and DPU were dissolved in DMSO to prepare stock solutions. Ten thousand A549 or Vero-E6 cells were seeded in each well of a 96-well optical-bottom plate (Greiner #655090). Next day, cells were washed with infection medium. A549 cells were infected with IAV (X-31 and WSN) and Vero-E6 cells were infected with SARS-CoV-2 (D614G) at MOI: 0.2-0.6 in presence of DPUDs (10 μM), DPU (10 μM), BafA1 (50 nM), regorafenib (10 μM), sorafenib (10 μM), favipiravir (10 μM), HCQ (10 μM), and niclosamide (10 μM) Ten h.p.i. (IAV) or 24 h.p.i. (SARS-CoV-2), cells were washed with phosphate buffered saline (PBS, pH 7.4) and fixed with 4% formaldehyde in PBS for 20 min at room temperature (RT). Indirect immunofluorescence (IIF) was performed with anti-NP (IAV) and anti-N (SARS-CoV-2) antibodies using previously-described protocols (Banerjee I et al.,. (2013) & Daly J. L. et al.,. (2020). Nuclei were stained with Hoechst. The infection assays were performed in triplicate. Nine image fields were randomly selected in each well, and automatically imaged using a 20× objective with Yokogawa CQ1 high-content, dual-spinning disk, confocal microscope with maximum intensity projection of five Z-stacked images. Percentage infection was quantified by image analysis pipeline setup in CellProfiler (version 2.2.0) and KNIME (version 3.7.2). Data was plotted in Graphpad Prism 9 and statistical analysis was carried out using a one-way ANOVA with multiple comparisons.

### IC50 and CC50 Calculation

To determine the IC50 values of the DPUDs, first, A549/Vero-E6 cells were seeded in optical bottom 96 well plates. Next day, DPUDs were serially diluted (10 μM to 10 nM) in DMSO, and cells were pre-treated with respective dilutions of the compounds in infection medium at 37° C. for 1 h. Following pre-treatment, the cells were infected with the viruses (IAV in A549 cells and SARS-CoV-2 in Vero-E6 cells) in presence of different concentrations of DPUDs, and incubated at 37° C. for 10 h (IAV) or 24 h (SARS-CoV-2) in CO₂ incubator. After fixing the cells, IIF was performed as described above to detect infected cells. IC50 was determined using non-linear regression function and plotted [inhibitor] vs. normalised response-variable slope. To determine the CC50 values of the DPUDs in A549 and Vero-E6 cells, colorimetric cytotoxicity assay was performed using LDH cytotoxicity assay kit (CytoTox 96 Non-Radioactive Cytotoxicity Assay, Promega) as per the manufacturer's protocol. Briefly, ten thousand A549 or Vero-E6 cells were seeded in 96-well plates. Next day, the cells were treated with serially diluted (500 μM to 1 μM) DPUDs in cell culture medium. LDH was measured 24 h post-treatment from the supernatant at 490 nm using Multiskan GO plate reader (Thermo Scientific). CC50 was calculated using non-linear regression function and plotted [inhibitor] vs. normalised response-variable slope using Graphpad Prism 9.

### qRT-PCR for Measuring SARS-CoV-2 Gene Expression

To detect SARS-CoV-2 genes, Vero-E6 cells (1×104) were seeded in a 96-well plate. Next day, the cells were pre-treated with 1 μM of respective DPUDs in serum-free DMEM for one hour at 37° C., followed by infection with SARS-CoV-2 (MOI0.5) in presence DPUDs for 1 h. Medium was replaced with virus-free complete medium with DPUDs, and supernatant was collected at 24 h.p.i. SARS-CoV-2 E and N gene expression was detected and quantified using Quantiplus Multiplex (Huwel Lifesciences) or DiAGSure (GCC Biotech) COVID-19 detection kit. The analysis of the virus inactivation was based on the quantification of viral RNA (cycle threshold [Ct] profile) present in the culture supernatant.

### Western Blotting and Viral Plaque Assay

A549 cells were seeded in a 6 well plate (2×105 cells/well) 24 h prior to infection. Cells were pre-treated with DPUDs dissolved in DMSO (10 μM) or DMSO alone at 37° C. for 1 h. Following pre-treatment, the cells were infected with IAV X-31 or WSN (MOI 0.1) and incubated for 24 h at 37° C. in a humidified chamber with an atmosphere of 5% CO₂. The supernatant was collected and centrifuged to remove cellular debris. For plaque assay, MDCK cells were seeded at a density of 2×10⁵ in a 24 well plate. Next day, the supernatant containing viruses was serially diluted till 10⁻⁶ dilution, and MDCK cells were inoculated with supernatants for 1 h. Cells were washed with PBS, and they were overlaid with infection medium with 0.8% low melting agarose (Genaxy) and 1 μg/ml TPCK trypsin (Merck). The cells were fixed at 48 h.p.i. with 4% formaldehyde for 1 h. Agarose plug was carefully removed and the cells were stained with crystal violet solution (Himedia) for 15 min. Excess stain was removed by holding the plate under controlled flowing water. Viral plaques were counted, and PFU/ml was calculated. For western blotting, cells infected with IAV X-31 or WSN were lysed using RIPA buffer (CSH protocols) in presence of a protease inhibitor cocktail (Merck). The whole-cell lysate was vortexed at 4° C. for 15 min, followed by centrifugation at 12000 rpm at 4° C. for 30 min. The clarified lysate was mixed with 6× SDS loading dye and treated at 95° C. for 10 min. SDS-PAGE was performed, and the proteins were transferred on nitrocellulose membrane (Bio-Rad). IAV hemagglutinin (HA) was stained with rabbit anti-HA (Pinda) antibody, and the loading control, GAPDH, was stained rabbit anti-GAPDH antibody (CST #2118). HRP-labelled secondary antibodies (CST) were illuminated with ECL substrate mix (Bio-Rad), and the blot was developed using ImageQuant LAS 4000 system.

### Animal Experiments

C57BL/6 mice were obtained from the Jackson Laboratory, USA, and bred in an individual ventilated caging system at the Small Animal Facility for Experimentation (SAFE), IISER Mohali. Institutional Animal Ethics Committee (IAEC) of IISER Mohali set up by the Committee for the Purpose of Control and Supervision of Experiments on Animals (CPCSEA), Which is established under Chapter 4, Section 15(1) of the Prevention of Cruelty to Animals Act 1960. IAEC approved the experimental animal protocols. The protocol number was IISERM/SAFE/PRT/2021/003. All operations were carried out in exact accordance with the authorised guidelines. For IAV infection, 6-10-week-old C57BL/6 mice were anesthetized using a ketamine-xylazine cocktail. The test level of anesthesia was examined by pinch reflex. Mice were intranasally infected with 1000 PFU of IAV (WSN). The investigator adequately monitored anesthetic recovery. After 24 h.p.i., the mice were intraperitoneally administered with DPUDs or oseltamivir acid dissolved in DMSO or an equal volume of DMSO dissolved in PBS (1 mg/kg of mouse body weight) twice a day for the next 5 days. Bodyweight was monitored every day. After ten days, animals were euthanized using CO₂ asphyxiation. Lungs and spleens were excised and homogenized in DMEM. RNA was isolated from tissue homogenate using Tri reagent (Merck) and qRT-PCR was performed for detection of IAV NP expression using the primers: 5′GGAGTCTTCGAGCTCT3′ (fwd) and 5′TTTTTTTTTTTTTTTTTTTAATTGTCGTACTCCT3′ (rev). Plaque assay was performed using the lung homogenate supernatant in MDCK cells using the method described above.

### Virus Resistance Assay

Twenty-four hours prior to infection, MDCK cells (1×105) were seeded in a 24-well plate. Cells were pre-treated with 1 μM DPUD-1/oseltamivir acid dissolved in DMSO and an equal volume of DMSO in the infection media for 1 h at 37° C. Cells were infected with IAV WSN (MOI: 0.1) for 1 h in presence of the compounds. Cells were washed with 1xPBS and replaced with infection medium with TPCK trypsin (1 μg/ml) and the compounds. After 48-72 h.p.i., the supernatant was collected and clarified for cell debris by centrifugation, and 1:10 dilution of the supernatant was used for the next round of infection. Similarly, viruses were passaged nine more times, and after the 10th passage, viral load was quantified by plaque assay.

### IAV Cellular Entry Assays

IAV cellular entry assays (virus binding, endocytosis, HA acidification, M1 uncoating, and vRNP nuclear import) were performed as follows: Briefly, A549 cells were seeded in 96-well plate and after they reached 70-80% confluency, IAV entry assays were performed. In every assay, the cells were pre-treated with DPUDs dissolved in DMSO (10 μM) or DMSO at 37° C. for 1 h. In endocytosis assay, CPZ (70 μM) was added as positive control. BafA1 (50 nM) was included as positive control in the HA acidification, M1 uncoating, and vRNP nuclear import assays. In the virus binding assay, 0.25 μL of purified X-31 (TCID50: 1×108) viruses were added in each well of a 96-well plate, and the viruses were allowed to bind to cells at 4° C. for 1 h. Unbound viruses were removed by multiple washes with ice-cold PBS. Cells with the bound viruses were fixed with 4% formaldehyde on ice for 10 minutes, and they were further incubated with the fixative at RT for 10 min. In the endocytosis and HA acidification assays, 0.25 μL of purified X-31 was added in each well, whereas, 0.50 μL of the virus was added in each well for the M1 uncoating and vRNP nuclear import assays. Viruses were allowed to enter cells in presence of DPUDs dissolved in DMSO (10 μM) at 37° C. for different time durations: 30 min (endocytosis), 1 h (HA acidification), 2.5 h (uncoating), and 4 h (vRNP nuclear import). After virus entry, the cells were fixed with 4% formaldehyde at RT for 20 min. All the virus entry assays were performed in presence of cycloheximide (1 mM) to prevent synthesis of new viral proteins. In the endocytosis assay, first, a saturating concentration (1:1000) of a rabbit polyclonal anti-HA antibody (Pinda), diluted in a non-permeabilizing solution (1% BSA and 5% FBS in PBS) was used to block all the HA epitopes present on the surface-bound virus. Upon permeabilization with saponin-containing buffer (0.1% saponin, 1% BSA, and 5% FBS in PBS), a mouse monoclonal anti-HA1 antibody (H3SKE) (1:100) was used to detect the internalized virus. The acid conformation of HA was detected using an acid conformation-specific mouse monoclonal antibody (A1) (1:1000). Viral M1 was stained with HB64 antibody (1:250), and NP was stained with HB65 antibody (1:30). Appropriate AF-conjugated secondary antibodies (Invitrogen) were used to detect the IAV proteins, and the nucleus was stained with Hoechst (Invitrogen). Imaging was done with 40× objective using Yokogawa CQ1 high-content, dual-spinning disk confocal microscope with maximum intensity projection of five Z-stacked images.

### Pseudotyped SARS-CoV-2 Preparation and Entry Inhibition Assay

Viral entry inhibition assay was performed with pseudotyped SARS-CoV-2 as previously described (Schmidt, F. et al. (2020). Briefly, HEK293T cells were transfected with plasmid DNA pHIV-1 NL4·3Δenv-Luc and Spike-expressing construct. The spike plasmid expresses spike protein of D614G or Delta or Omicron variants. In all the three spike constructs, D614G mutation was present, which enhances the infectivity of the pseudovirus. The transfection was done by using Profection mammalian transfection kit (Promega Inc.) following the instructions of the kit manual. Pseudovirus decorated with SARS-CoV-2 spike proteins were harvested 48 h after transfection, clarified by filtration using 0.45 μm filters, and stored at −80° C. until use. 293T-hACE-2 (BEI resources, NIH, #NR-52511) cells expressing the ACE2 receptor were cultured in complete DMEM supplemented with 5% FBS. Entry-inhibition assays with the inhibitors were done in three replicates. The pseudovirus was incubated with serially diluted DPU/DPUDs/niclosamide dissolved in DMSO and an equal volume of DMSO in a total volume of 100 μL for 15 min at 37° C. The 293T-hACE2 cells were then trypsinised, and ten thousand cells/well were added to make up the final volume of 200 μL/well. The plates were further incubated for 48 hs in a humidified incubator at 37° C. with 5% CO₂. After incubation of cells, 140 μL cell culture media was removed, and 50 μL nano-Glo luciferase substrate (Promega Inc.) along with the lysis buffer was added. After lysing cells for 2-3 minutes, 80 μL lysate was transferred to white plates, and luminescence was measured by using Cytation-5 multi-mode reader (BioTech Inc.) DMSO normalised RLU were plotted using GraphPad Prism.

### Plasma Membrane Bypass Infection Assay

Plasma membrane (PM) bypass infection assay was carried out as described below. Briefly, A549 cells (1×104) were seeded in two optical bottom 96-well plates. Next day, cells in one plate were pre-treated with DPUDs dissolved in DMSO (10 μM) or DMSO at 37° C. Following pre-treatment, viruses (0.5 μl of purified X-31 in 50 μL infection medium/well) were allowed to bind to the PM at 4° C. for 1 h in presence of DPUDs dissolved in DMSO (10 μM). Pre-warmed fusion medium (DMEM with citrate buffer, pH 5.0) or STOP medium (DMEM with 50 mM HEPES, pH 7.4 and 20 mM NH₄Cl) was added to cells and quickly shifted to 37° C. for 3 min. Cells were rapidly washed with stop medium and incubated in the STOP medium for additional 10 h. The same assay was performed in the other plate, but without addition of DPUDs. One-hour post-fusion, cells were incubated with stop medium with DPUDs (10 μM) for 9 h. Cells were then washed with PBS, fixed with 4% formaldehyde, and processed for IIF staining against IAV NP and imaging as described above.

### Time of Addition Assay

A549 or Vero-E6 cells (1×104) were plated on an optical-bottom 96-well plate. After the cells reached 70-80% confluency, they were either pre-treated with DPUDs dissolved in DMSO (10 μM and 1 μM in A549 cells and Vero-E6 cells, respectively) or DMSO for 2 h (−2 h) before infection with IAV (WSN) sir SARS-CoV-2 (D614G), or treated with the compounds added at different time points during infection: 0 h, 2 h, 4 h, 6 h, and 8 h. Cells were fixed 10 h.p.i and 24 h.p.i upon infection with IAV and SARS-CoV-2, respectively. IIF was performed as described above for the detection of infected cells.

### Live-Cell Imaging to Monitor RV Internalization

To fluorescently label IAV (X-31) particles for live-cell imaging, the lipophilic dye SP-DiOC18(3) (Invitrogen) was used. First, the dye was dissolved in ethanol to prepare 1.8 mM stock solution. Next, 100 μL purified IAV (X-31, TCID50: 1.0×108) was diluted in 1400 μL PBS. A final concentration of 2 μM SP-DiOC18(3) from the stock of 1.8 mM was used to label the virus diluted in a total volume of 1500 μL (100 μL virus+1400 μL PBS). The dye was slowly added to the virus while vortexing at low speed. Following dye addition, the virus was incubated at RT for 1 h on a rotary mixer. The labelled virus was passed through a 0.2 μm filter and used fresh for live-cell imaging. For live-cell imaging, A549 cells (1×104) were seeded on an eight well μ-Slide (Ibidi). The cells were transfected with Rab5-RFP (Addgene #14437) using lipofectamine LTX (Invitrogen), 16-18 h prior to live-cell imaging. One hour before live-cell imaging, the cells were incubated with DPUD-1 dissolved in DMSO (10 μM) or DMSO alone. After pre-treatment, the medium was replaced with serum-free medium (100 μM) containing SP-DIOC18(3)-labelled virus particles and incubated at 37° C. for 15 min in presence of DPUD-1 dissolved in DMSO or DMSO alone, allowing virus entry. Next, the cells were washed with serum-free medium to remove unbound viruses and replaced with fresh serum-free medium. Imaging was done using alpha plan apochromat 100×/1.46 N.A. oil objective in Zeiss LSM 980 with Airy-scan 2 system.

### EGF, Transferrin and Cholera Toxin B Uptake Assays

To examine the effect of DPUDs in clathrin- or caveolin-mediated endocytosis, EGF, transferrin and cholera toxin B (CTXB) uptake assays were performed. A549 cells (1×104) were seeded in a 96-well optical-bottom plate. Next day, the cells were incubated with serum-free medium 2 h prior to the start of the experiments. Following incubation with serum-free medium, the cells were pre-treated with DPUDs dissolved in DMSO (10 μM) or DPU dissolved in DMSO (10 μM) or MβCD (10 mM) or DMSO in serum-free medium for 1 h, or with CPZ (70 μM) for 10 min at 37° C. Cells were pulsed with EGF-AF488 (30 ng/ml) (Invitrogen) for 30 min, transferrin-AF488 (30 μg/ml) (Invitrogen) for 10 min and with CTXB-AF488 (1 μg/mL) (Invitrogen) for 15 min in presence of the compounds in complete medium (with serum) at 37° C. After respective time durations, cells were washed twice with PBS, followed by treatment with acidic buffer (150 mM NaCl and 50 mM Glycine, pH 3.0) for 2 min to remove the surface-hound ligands. Cells were again washed with PBS and fixed with 4% formaldehyde for 20 min at RT. Nucleus was stained with Hoechst diluted in PBS (1:10000). High-content microscopy and image analysis were performed as described above.

### Vesicular Acidification Assay

A549 cells (1×104) were seeded in an optical-bottom, 96-well plate. Next day, the cells were pre-treated with DPUDs dissolved in DMSO (10 μM) or DPU dissolved in DMSO (10 μM) or BafA1 (50 nM) or DMSO at 37° C., for 1 h. Next, the cells were incubated with LysoTracker Red DND-99 (1 μM) (Invitrogen #L7528) in presence of the compounds at 37° C. for 1 h. The cells were washed with 1×PBS and fixed with 4% formaldehyde. The nucleus was stained with Hoechst. High-content microscopy and image analysis were performed as described above.

### In Vitro Calcein Release Assay

Calcein release assay was performed as previously described (Chattopadhyay et. Al. (2002). Briefly, 100 mM calcein (Merck) was prepared with 1 mg/ml phospholipid mix (Asolectin (90): cholesterol (10)) in an internal buffer (20 mM Tris-HCl, 150 NaCl, pH 8.0). Purified calcein-encapsulated LUV was prepared in 2 ml reaction mix with an external buffer (20 mM Tris-HCl, 150 NaCl, pH 8.0). DPUDs dissolved in DMSO (10 μM) or DPU dissolved in DMSO (10 μM) or DMSO were added 30 sec post-acquisition start time. Sodium deoxycholate (6 mM) was used as a positive control for calcein release. Percentage calcein release vs. time was plotted using Graphpad Prism 9.

### Image Acquisition and Analysis

Automated high-content image acquisition was performed using a 20× or 40× objective using the Yokogawa CQ1 dual-spinning disk confocal microscope. Nine (3×3) images were acquired from each well of an optical-bottom 96-well plate for each fluorescence channel, typically resulting in the counting of 3,000-7,000 cells in the samples. High-resolution images were acquired using Leica TCS SP8 confocal microscope or Zeiss LSM 980 with Airyscan 2 system. Minor image processing (cropping, brightness and contrast adjustments) were done in ImageJ (2.0.0-rc-69/1.52p) and Adobe Photoshop (21.2.12) for better visualization. Confocal images from multiple planes were Z-stacked by maximum intensity projection. Percentage infection was quantified by detection of cell nuclei and NP (IAV)- or N (SARS-CoV-2)-expressing cells by an image analysis pipeline created in Cell Profiler (version 2.2.0) and KNIME (version 3.7.2). IAV uncoating and vRNP nuclear import analysis were carried out using the same image analysis module. Integrated density (product of mean fluorescence intensity and area) was calculated for the other assays.

### Statistical Analysis

Data are represented as the mean±SD. For all analyses, multiple independent experiments (n≥3) were carried out. Statistical analysis was performed using Graphpad Prism 9, and the P value was calculated by one-way ANOVA with multiple comparisons.

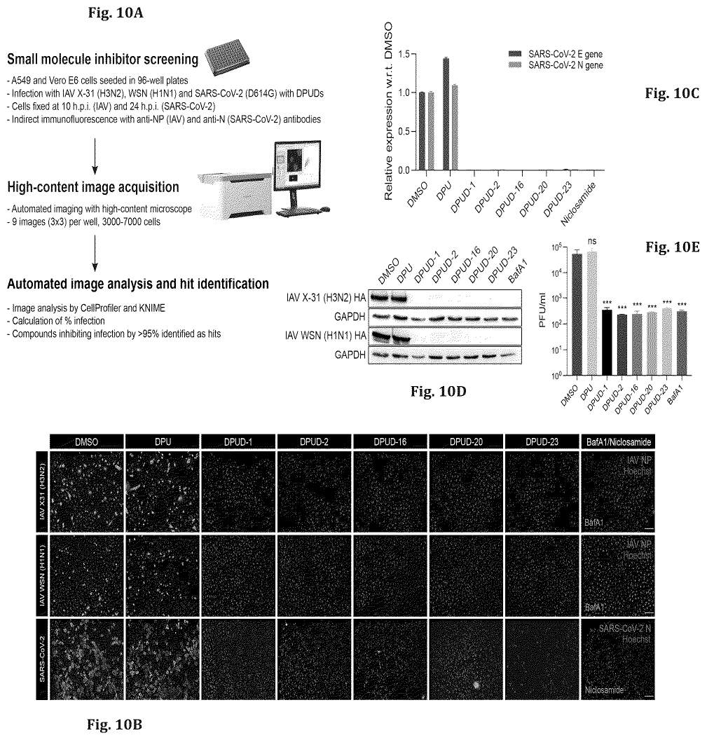

High-content screens of synthesized compounds against IAV A/X-31 (H3N2) and SARS-CoV-2 D614G strains using cell-based infection assays were performed. The infection assay was based on the detection of IAV nucleoprotein (NP) and SARS-CoV-2 N protein in human alveolar epithelial cell line (A549) and African green monkey kidney cell line (Vero-E6), respectively (FIG. 10a), 1,3-diphenylurea derivative (DPUD), 1,3-bis(3,5-bis(trifluoromethyl)phenyl)urea (DPUD-1), were identified to reduce the infections of both IAV and SARS-CoV-2 by greater than 99% at 10 M concentration. 22 additional DPUDs (Table 1) have been synthesized and the infection screens using two IAV strains (A/X-31, H3N2 and A/WSN/1933, H1N1) and SARS-CoV-2 D614G strain were performed. Since DPUDs were derivatized from 1,3-diphenylurea (DPU), DPU was also included in the infection screen to examine if it had any effect in the viral infections. Dimethyl sulfoxide (DMSO) has been employed as the negative control in the infection screens and subsequent experiments. In the IAV infection screens, regorafenib and sorafenib, urea-based kinase inhibitors that block virus fusion, and bafilomycin A1 (BafA1), a highly potent and selective inhibitor of vacuolar H+-ATPases that inhibit infection by blocking endosomal acidification have also been employed. In the SARS-CoV-2 infection screen, favipiravir, niclosamide and hydroxychloroquine (HCQ) have been employed as positive controls as these compounds were previously found to be effective against SARS-CoV-2 (Drozdzal, S. et, al. (2021).

TABLE 1

| | | Mol. | |
|---|---|---|---|
| | | Formula | |
| | | (Mol. | |
| Compounds | Structure | Weight) | IUPAC |
| DPU | | C₁₃H₁₂N₂O | 1,3-diphenylurea |
| | | (212.0950) | |
| DPUD-1 | 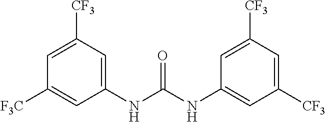 [SMILES: O=C(Nc1cc(C(F)(F)F)cc(C(F)(F)F)c1)Nc1cc(C(F)(F)F)cc(C(F)(F)F)c1] | C₁₇H₈F₁₂N₂O (484.0445) | 1,3-bis(3,5- bis(trifluoromethyl)phenyl) urea |
| DPUD-2 | | C₁₅H₁₀F₆N₂O | 1,3-bis(4- |
| | | (348.0697) | (trifluoromethyl)phenyl)urea |
| DPUD-3 | | C₁₅H₁₀F₆N₂O | 1,3-bis(2- |
| | | (348.0697) | (trifluoromethyl)phenyl)urea |
| DPUD-4 | | C₁₃H₁₀F₂N₂O | 1,3-bis(4- |
| | | (248.0761) | fluorophenyl)urea |
| DPUD-5 | | C₂₇H₂₄N₂O | 1,3-bis(4- |
| | | (392.1889) | benzylphenyl)urea |
| DPUD-6 | | C₁₃H₁₀Br₂N₂O | 1,3-bis(3- |
| | | (367.9160) | bromophenyl)urea |
| DPUD-7 | | C₁₃H₁₀Br₂N₂O | 1,3-bis(4- |
| | | (367.9160) | bromophenyl)urea |
| DPUD-8 | | C₁₃H₁₀Cl₂N₂O | 1,3-bis(4- |
| | | (280.0170) | chlorophenyl)urea |
| DPUD-9 | | C₁₇H₂₀N₂O | 1,3-bis(2,4- |
| | | (268.1576) | dimethylphenyl)urea |
| DPUD-10 | | C₁₇H₂₀N₂O | 1,3-bis(2,5- |
| | | (268.1576) | dimethylphenyl)urea |
| DPUD-11 | | C₁₃H₁₀Cl₂N₂O | 1,3-bis(2- |
| | | (280.0170) | chlorophenyl)urea |
| DPUD-12 | | C₁₇H₂₀N₂O₅ | 1,3-bis(3,4- |
| | | (332.1372) | dimethoxyphenyl)urea |
| DPUD-13 | 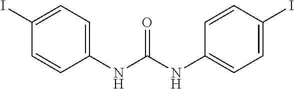 [SMILES: O=C(Nc1ccc(I)cc1)Nc1ccc(I)cc1] | C₁₃H₁₀I₂N₂O (463.8882) | 1,3-bis(4- iodophenyl)urea |
| DPUD-14 | 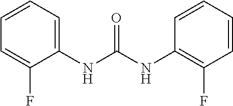 [SMILES: O=C(Nc1ccccc1F)Nc1ccccc1F] | C₁₃H₁₀F₂N₂O (248.0761) | 1,3-bis(2- fluorophenyl)urea |
| DPUD-15 | 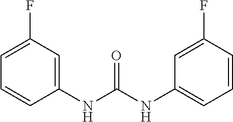 [SMILES: O=C(Nc1cccc(F)c1)Nc1cccc(F)c1] | C₁₃H₁₀F₂N₂O (248.0761) | 1,3-bis(3- fluorophenyl)urea |
| DPUD-16 | 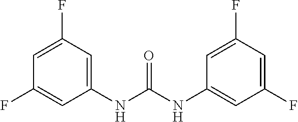 [SMILES: O=C(Nc1cc(F)cc(F)c1)Nc1cc(F)cc(F)c1] | C₁₃H₈F₄N₂O (284.0573) | 1,3-bis(3,5- difluorophenyl)urea |
| DPUD-17 | 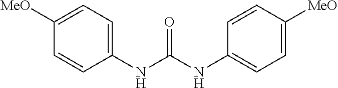 [SMILES: COc1ccc(NC(=O)Nc2ccc(OC)cc2)cc1] | C₁₅H₁₆N₂O₃ (272.1161) | 1,3-bis(4- methoxyphenyl)urea |
| DPUD-18 | 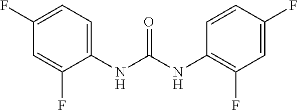 [SMILES: O=C(Nc1ccc(F)cc1F)Nc1ccc(F)cc1F] | C₁₃H₈F₄N₂O (284.0573) | 1,3-bis(2,4- difluorophenyl)urea |
| DPUD-19 | 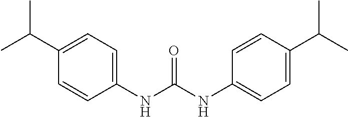 [SMILES: CC(C)c1ccc(NC(=O)Nc2ccc(C(C)C)cc2)cc1] | C₁₉H₂₄N₂O (296.1889) | 1,3-bis(4- isopropylphenyl)urea |
| DPUD-20 | 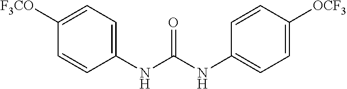 [SMILES: O=C(Nc1ccc(OC(F)(F)F)cc1)Nc1ccc(OC(F)(F)F)cc1] | C₁₅H₁₀F₆N₂O₃ (380.0596) | 1,3-bis(4- (trifluoromethoxy)phenyl) urea |
| DPUD-21 | 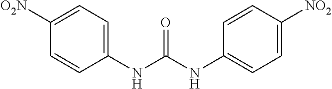 [SMILES: O=C(Nc1ccc([N+](=O)[O-])cc1)Nc1ccc([N+](=O)[O-])cc1] | C₁₃H₁₀N₄O₅ (302.0651) | 1,3-bis(4- nitrophenyl)urea |
| DPUD-22 | 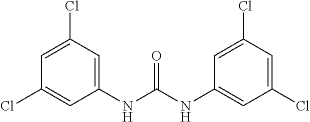 [SMILES: O=C(Nc1cc(Cl)cc(Cl)c1)Nc1cc(Cl)cc(Cl)c1] | C₁₃H₈Cl₄N₂O (347.9391) | 1,3-bis(3,5- dichlorophenyl)urea |
| DPUD-23 | 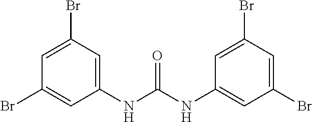 [SMILES: O=C(Nc1cc(Br)cc(Br)c1)Nc1cc(Br)cc(Br)c1] | C₁₃H₈Br₄N₂O (523.7370) | 1,3-bis(3,5- dibromophenyl)urea |

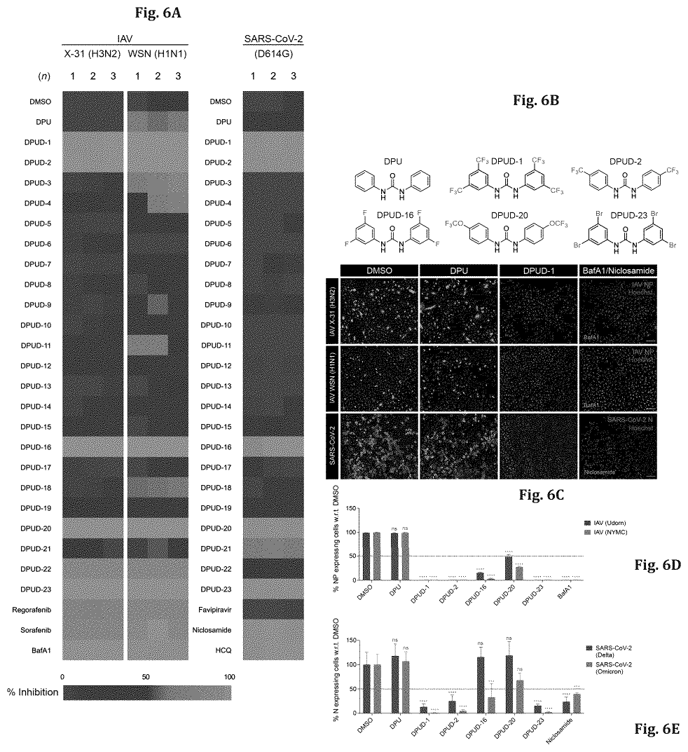

The effectiveness of DPUD-1 against both the viruses have been reconfirmed in infection screens. Four additional DPUDs that blocked IAV and SARS-COV-2 infections by 95-100% have been identified: 1,3-bis(4-(trifluoromethyl)phenyl)urea (DPUD-2), 1,3-bis(3,5-difluorophenyl)urea (DPUD-16), 1,3-bis(4-trifluoromethoxy)phenyl)urea (DPUD-20), and 1,3-bis(3,5-dibromophenyl)urea (DPUD-23) (Table-2; FIG. 6a-c), FIG. 10b). However, DPU did not show any inhibitory effect against any of the viruses.

TABLE 2

| | | Mol. | |
|---|---|---|---|
| | | formula | |
| | | (Mol. | |
| Compounds | Structure | Weight) | IUPAC |
| DPTUD | | (500.0217) | 1,3-bis(3,5- |
| | | | bis(trifluoromethyl)phenyl) |
| | | | thiourea |
| DPUD- 1 | 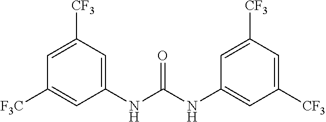 [SMILES: O=C(Nc1cc(C(F)(F)F)cc(C(F)(F)F)c1)Nc1cc(C(F)(F)F)cc(C(F)(F)F)c1] | C₁₇H₈F₁₂N₂O (484.0445) | 1,3-bis(3,5- bis(trifluoromethyl)phenyl)urea |
| DPUD- | | C₁₅H₁₀F₆N₂O | 1,3-bis(4- |
| 2 | | (348.0697) | (trifluoromethyl)phenyl)urea |
| DPUD- | | C₁₃H₈F₄N₂O | 1,3-bis(3,5- |
| 16 | | (284.0573) | difluorophenyl)urea |
| DPUD- | | C₁₅H₁₀F₆N₂O₃ | 1,3-bis(4- |
| 20 | | (380.0596) | (trifluoromethoxy)phenyl)urea |
| DPUD- | | C₁₃H₈Br₄N₂O | 1,3-bis(3,5- |
| 23 | | (523.7370) | dibromophenyl)urea |

The heatmaps of the screen results showed almost similar patterns of inhibition of both the viruses (FIG. 6a). The viral titre of SARS-CoV-2 in the supernatant of the infected cells, treated with the hit compounds (DPUD-1, -2, -16, -20 and -23) has been quantified. 99-100% decrease in viral titres was found in the supernatants of the DPUD-treated cells as compared to DMSO control (FIG. 10c). Western blotting against the IAV HA protein and IAV plaque assay in Madin-Darby canine kidney (MDCK) cells further confirmed robust potency of the compounds in blocking IAV (WSN) infection (FIG. 10d, 10e).

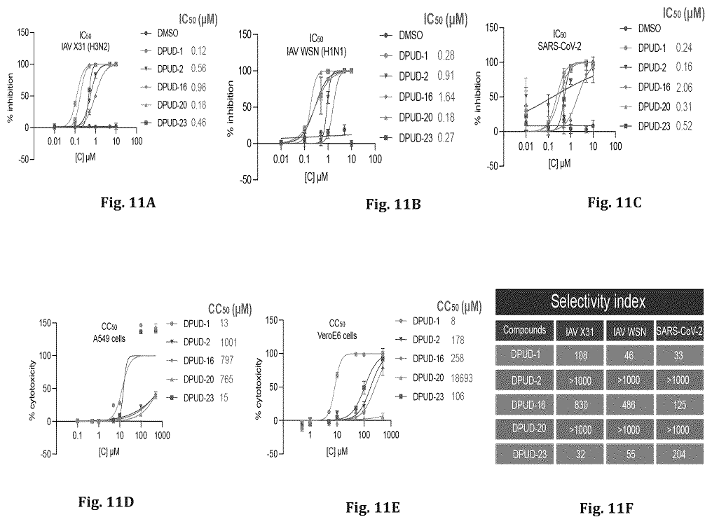

The IC50 values for all the compounds have been determined and it was found that except for DPUD-16, all the other compounds showed IC50 values <1.0 M (FIG. 11a-11c). The CC50 values on A549 and Vero-E6 cells (FIG. 11d-11e) have been determined by which the selectivity indices (SIs) of all the tested hit compounds have been computed and it was found to be >30 SI values, highlighting their potential as antiviral drugs (FIG. 11f).

To check the breath of inhibitory effect of DPUDs, their effect against additional strains of IAV (A/Udorn/1972, H3N2, and A/New York/55/2004, H3N2) and SARS-CoV-2 (Delta, B.1.617.2 and Omicron, B1.1.529) were tested. DPUD-1, -2, and -23 were highly effective against all the tested virus strains (FIG. 6 d,e), highlighting their potential for development as broad-spectrum antiviral agents.

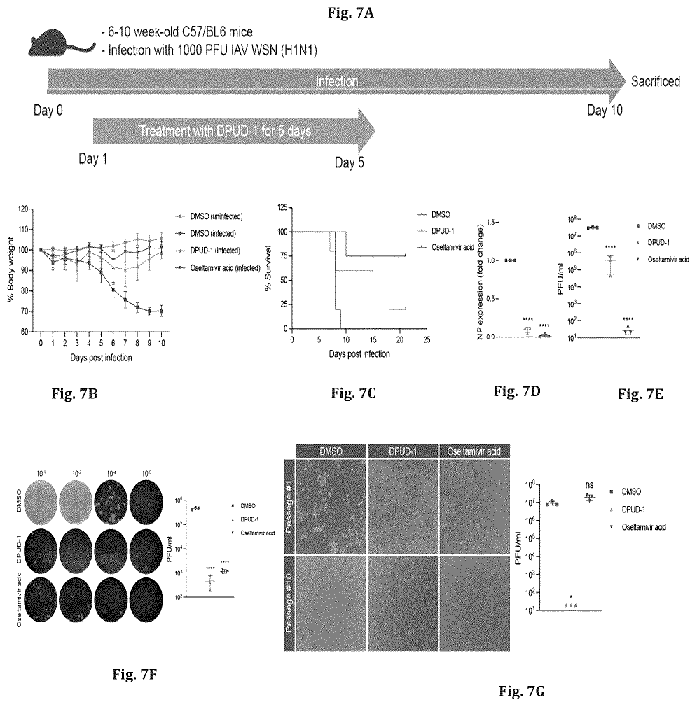

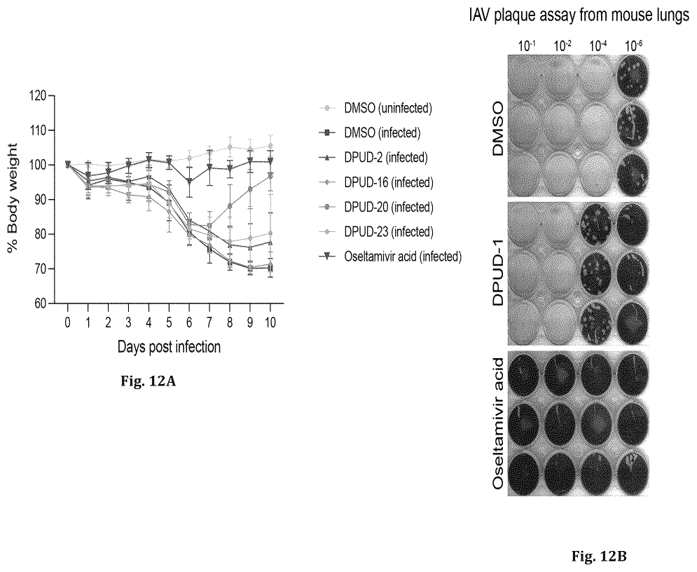

After confirming the antiviral potency of DPUDs in vitro, their effectiveness against IAV infection in mice has been tested. First, C57BL/6 mice were intranasally infected with 1000 plaque-forming units (PFUs) of IAV (WSN). After 24 hours, DPUDs dissolved in DMSO were administered intraperitoneally at 1 mg/kg of body weight twice daily for 5 days. There were six mice per group, and DMSO and oseltamivir acid were employed as negative and positive controls, respectively. Next, the body weights of the mice have been measured for 10 days post-infection (d.p.i). Among all DPUDs, DPUD-1 was the most effective in body weight recovery of the infected mice (FIG. 7a, b, FIG. 12a). Better survival of the DPUD-1-treated mice was also observed in comparison to DMSO controls till 21 d.p.i. (FIG. 7b).

Next, the lung viral titres of the mice sacrificed on the sixth day post-infection was determined by RT-PCR targeting the IAV NP gene and by virus plaque assay. A significant reduction in lung viral titres in the DPUD-1-treated mice as compared to DMSO controls (FIG. 7d,e, FIG. 12b) was observed. Also, in virus plaque assay, a significant reduction in IAV (WSN) titre upon DPUD-1 (1 M) treatment was observed. The virus titre in the DPUD-1-treated cells was comparable to the titre in cells treated with oseltamivir acid (1 M) (FIG. 7f).

Next, the barrier of DPUDs to viral resistance was assessed. IAV (WSN) strain in MDCK cells in presence of DPUDs (1 M), oseltamivir acid (1 M) and DMSO were passaged for 10 passages. After the tenth passage, the DPUD-1- and oseltamivir-passaged viruses were evaluated for their respective susceptibility and compared them with the DMSO-passaged viruses. Whereas the oseltamivir-passaged viruses showed similar titre as DMSO-passaged viruses, indicating that the viruses acquired resistance through serial passaging, the DPUD-1-passaged viruses showed >5 log reduction in viral titre (FIG. 7g). This suggests that DPUD-1 has significantly higher barrier to resistance than the approved anti-influenza drug, and the high resistance barrier further indicates that DPUD-1 possibly targets host processes that the virus exploits to establish productive infection rather than the virus-encoded factors.

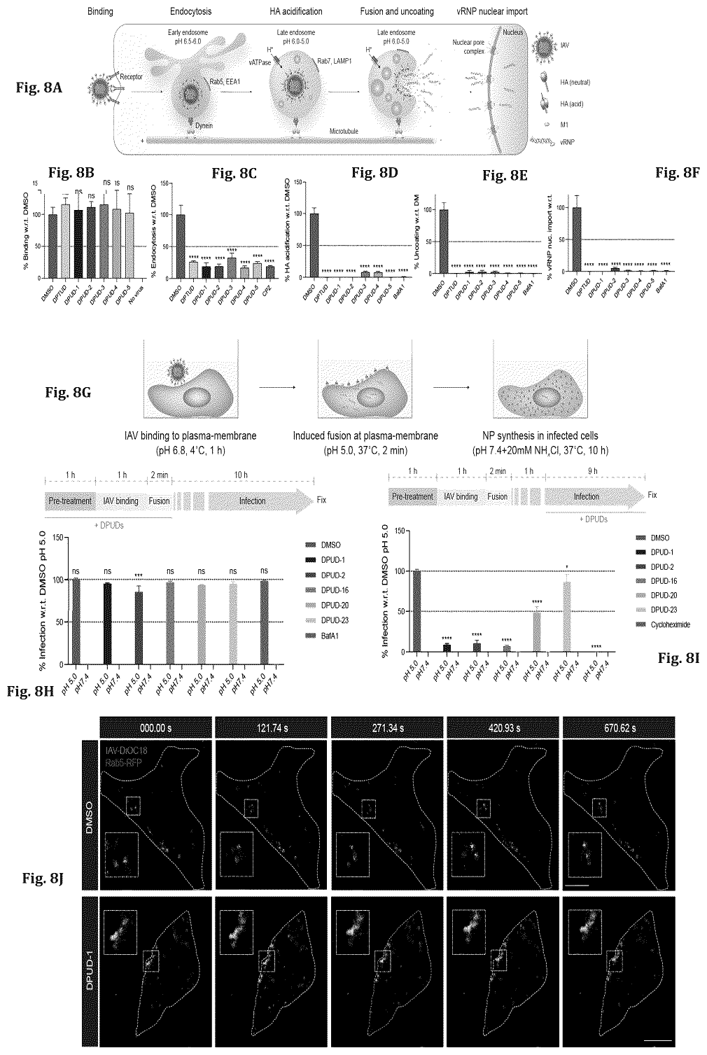

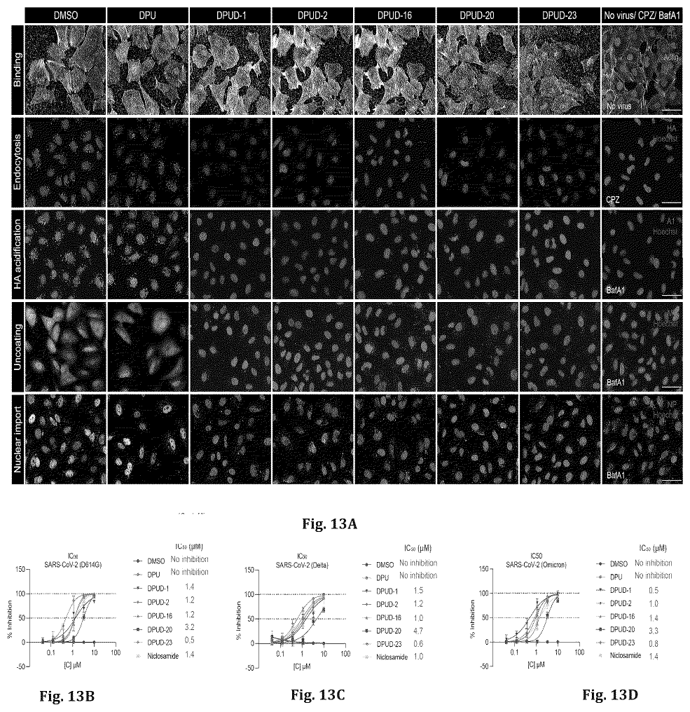

Next, the effect of DPUDs on IAV infection events in A549 cells were examined. Since virus entry represents the earliest stage of infection, the effect of DPUDs on IAV entry was checked. Cellular entry of IAV is a multi-step process (FIG. 8a), which begins with binding of the virus to the sialic acid-containing glycoproteins on the plasma membrane, followed by internalization through clathrin-mediated endocytosis and micropinocytosis. After sorting to acidic late endosomes in which, the low pH induces conformational changes in the HA protein, the virus fuses with the endosomal limiting membrane and uncoats, releasing the vRNPs into the cytosol. The vRNPs are then imported into the nucleus for replication and transcription. Using indirect immunofluorescence-based IAV entry assays, different steps of IAV entry upon DPUD treatment were probed. The cells were pretreated with DPUDs dissolved in DMSO (10 M) for 1 h, following which the IAV entry assays were performed using the X-31 strain in presence of the compounds (10 M). Although no significant difference in virus binding could be observed, endocytosis was blocked by 80% upon DPUD treatment. Expectedly, the subsequent steps of IAV entry i.e. HA acidification, uncoating and vRNP nuclear import were also robustly blocked (FIG. 8b-8f, FIG. 13a).

To confirm that DPUDs target IAV endocytosis, virus fusion with the plasma membrane (PM) was induced at pH 5.0, a process that allows direct delivery of vRNPs into the cytosol, bypassing endocytosis. Post-fusion, the cells were shifted to a pH 7.4 medium with ammonium chloride (NH₄Cl), ensuring that the observed infection signal was from the viruses fused at the PM, and not due to the viruses that still entered the cells via endocytosis (FIG. 8g). As control, a pH 7.4 fusion medium with NH₄Cl, which does not induce virus fusion was employed. After 10 h post-fusion, indirect immunofluorescence was performed to detect viral NP. Treatment with DPUDs dissolved in DMSO (10 M) till virus fusion resulted in 80-98% infection as compared to DMSO control (FIG. 8h). This indicated that the primary effect of DPUDs was on IAV endocytosis; if endocytosis is bypassed, NP synthesis could still happen in absence of DPUDs. Further, evaluation was done to find out whether the compounds had any additional effect in the later states of infection i.e. replication/transcription.

Using the same experimental design, virus fusion was induced at the PM after binding, and uncoating and vRNP release were allowed to happen for 1 h. This was followed by treatment with DPUDs for another 9 h. As positive control, cycloheximide was included to prevent expression of viral proteins. If DPUDs target any post-fusion event, NP expression would be affected. Interestingly, it was found that post-fusion DPUD treatment significantly blocked NP expression, indicating their role in viral gene expression inhibition (FIG. 8i). Since DPUDs primarily blocked IAV endocytosis, the movement of IAV particles during entry in live cells (A549) was monitored, treated with one of DPUDs (DPUD-1). mCherry-tagged Rab5, an early endosome marker, was overexpressed, and the viruses were labelled with a green lipophilic dye, SP-DiOC18(3). In real time imaging, the virus particles internalized successfully in the DMSO-treated cells and displayed rapid and directed movements. However, in presence of DPUD-1, majority of the virus particles failed to enter the cells and remained stuck on the PM in clusters, often colocalizing with a static pool of Rab5-positive vesicles (FIG. 8j).

Confirming the effect of DPUDs in IAV endocytosis, their effect on SARS-CoV-2 entry was also examined. To examine SARS-CoV-2 entry, pseudotyped, luciferase-encoding, and replication-defective HIV-119, expressing the D614G, Delta and Omicron variants of the spike (S) protein were employed. HEK 293T-hACE2 cells were infected with the pseudotyped viruses in presence of DPUDs and determined the IC50 values. The IC50 values for DPUDs, except DPUD-20, were 1.5 μM across all the pseudotyped SARS-CoV-2 variants, whereas the IC50 values for DPUD-20 were 4.7 μM (FIG. 13b). Taken together, it was demonstrated that DPUDs block cellular entry of both IAV and SARS-CoV-2.

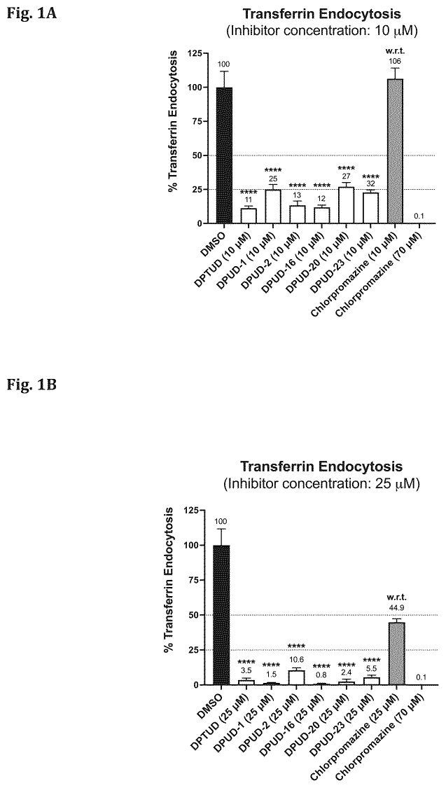

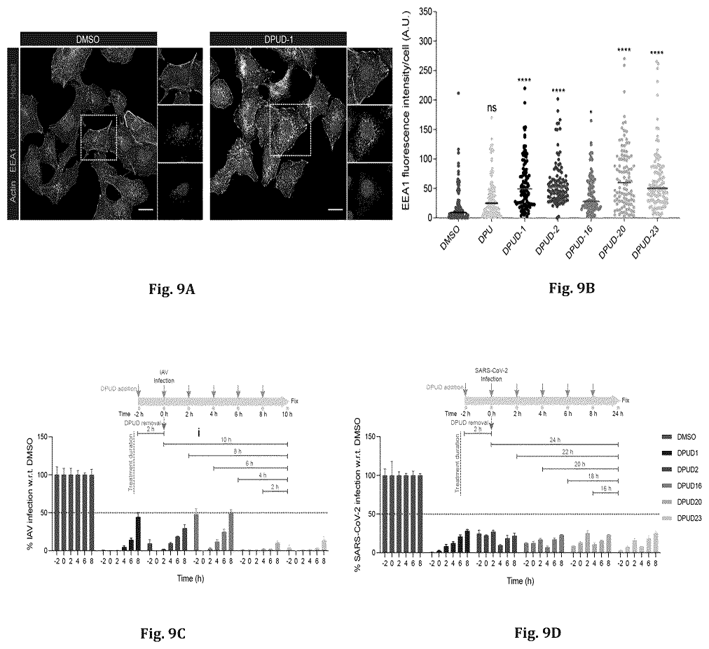

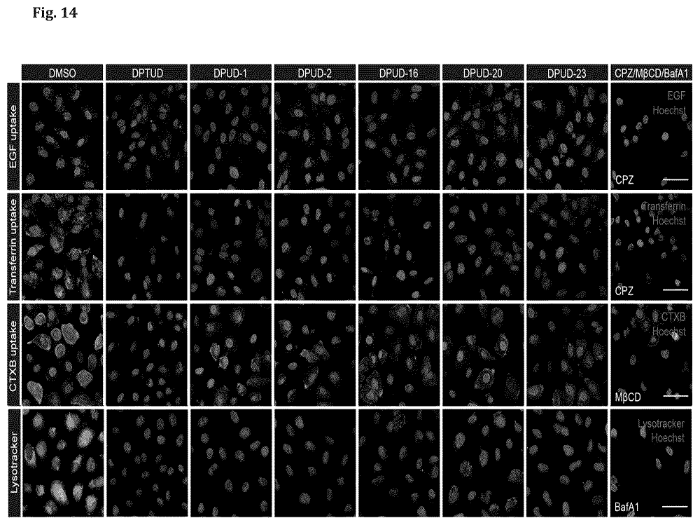

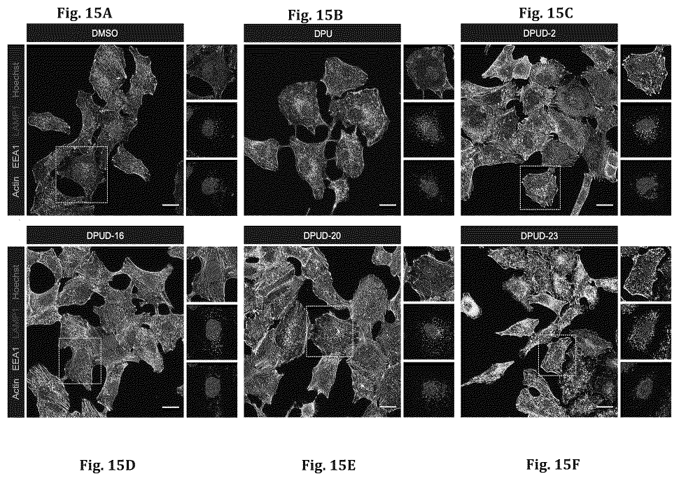

Inhibition of virus entry by DPTUD and DPUDs could either be virus-specific, or due to perturbation of the general endocytic pathways. To check the effect of DPTUD and DPUDs on transport of non-viral cargoes, cellular uptake of fluorescently-tagged epidermal growth factor (EGF), transferrin and cholera toxin B (CTXB) were probed, known to enter cells via multiple endocytic pathways. It was found that DPTUD and DPUDs significantly blocked their uptake and had most pronounced effect on transferrin endocytosis (FIG. 1,2, FIG. 14). Since disruption in endocytic machinery often leads to endosome maturation defects, indicated by elevated laminal pH, endosomal acidification was checked using LysoTracker, a fluorescent acidotropic probe that accumulates in acidic organelles. Treatment with DPTUD and DPUDs robustly inhibited organelle acidification as observed by LysoTracker-positive signal (FIG. 14). Confocal imaging of the DPUD-treated cells revealed significant upregulation of the early endosome specific marker EAA1, while dispersed signal was observed for LAMP1, a marker for late endosome/lysosome (FIG. 9 a,b, FIG. 15a-f).

To further examine DPUDs' effect in virus infection cycle, time of addition assays were performed. Cells were treated with DPUDs for different tune durations, IAV and SARS-CoV-2 infections were checked. Only two-hour pre-treatment of cells with DPUDs before virus addition was sufficient to block viral infections (FIG. 9 g,h). This indicated prior disruption of endocytic program, which was sustained even after DPUDs withdrawal, could arrest virus entry. Unexpectedly, DPUD treatment eight hours post addition of the viruses also reduced infection by 50%, suggesting interference with post-entry events, possibly with virus replication/transcription (FIG. 9c, d).

The details of the experiments explained above are explained in each of the Examples specified below:

### Example 1: Efficacy Comparison Between Chlorpromazine (Commercially Available Endocytosis Inhibitor) and Diphenyl Thiourea and Diphenyl Urea Derivatives in Blocking Transferrin Endocytosis

Human lung epithelial cells (A549) were seeded in a 96-well optical-bottom plate. When the cells reached 70-80% confluency, they were incubated with serum-free medium 2 h prior to the start of the experiment. Following incubation with the serum-free medium, the cells were pre-treated with the compounds dissolved in DMSO at indicated concentrations for 10 min at 37° C. Cells were pulsed with transferrin-AF488 (30 μg/ml) (Invitrogen) for 10 min in presence of the compounds in complete medium (DMEM with 10% FBS) at 37° C. Next, the cells were washed twice with PBS, followed by treatment with acidic buffer (150 mM NaCl and 50 mM Glycine, pH 3.0) for 2 min to remove the surface-bound ligands. Cells were again washed with PBS and fixed with 4% formaldehyde for 20 min at room temperature. Nucleus was stained with Hoechst diluted in PBS (1:10000). High-content microscopy and image analysis were performed to quantitate transferrin endocytosis (FIG. 1a,b). All data are represented as mean±SD. The P-value was determined using one-way ANOVA with multiple comparisons w.r.t. chlorpromazine (10 μM). ns: P>0.05, *P>0.05, **P<0.01, ***P<0.001, ****P<0.0001.

### Example-2: Efficacy Comparison Between Chlorpromazine and methyl-β-cyclodextrin (Commercially Available Endocytosis Inhibitors) and Diphenyl Thiourea and Diphenyl Urea Derivatives in Blocking EGF and Cholera Toxin B Endocytosis

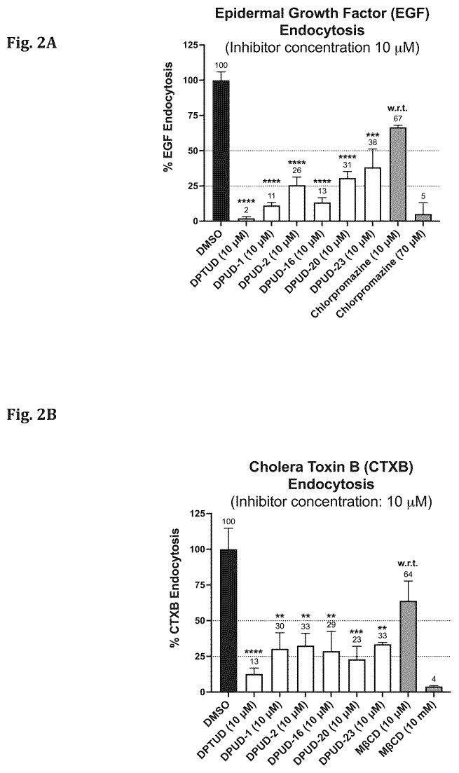

Human lung epithelial cells (A549) were seeded in a 96-well optical-bottom plate. When the cells reached 70-80% confluency, they were incubated with serum-free medium 2 h prior to the start of the experiment. Following incubation with the serum-free medium, the cells were pre-treated with the compounds (DPTUD and DPUDs) dissolved in DMSO at indicated concentrations for 10 min at 37° C. Cells were pulsed with EGF-AF488 (30 ng/ml) (Invitrogen) or cholera toxin B-AF488 (1 μg/mL) (Invitrogen) for 15 min in presence of the compounds in complete medium (DMEM with 10% FBS) at 37° C. Next, the cells were washed twice with PBS, followed by treatment with acidic buffer (150 mM NaCl and 50 mM Glycine, pH 3.0) for 2 min to remove the surface-bound ligands. Cells were again washed with PBS and fixed with 4% formaldehyde for 20 min at room temperature. Nucleus was stained with Hoechst diluted in PBS (1:10000). High-content microscopy and image analysis were performed to quantitate EGF and cholera toxin B endocytosis (FIG. 2a,b). All data are represented as mean±SD. The P-value was determined using one-way ANOVA with multiple comparisons w.r.t. chlorpromazine or methyl-β-cyclodextrin (10 μM). ns: P>0.05, *P<0.05, **P<0.01, ***P<0.001, ****P<0.0001.

### Example 3: Cytotoxicity Comparison Between Chlorpromazine (Commercially Available Endocytosis Inhibitor) and Diphenyl Thiourea and Diphenyl Urea Derivatives

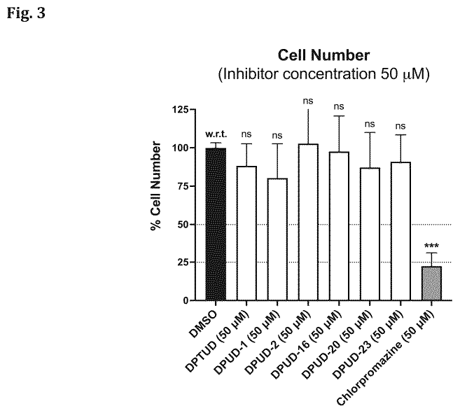

Human lung epithelial cells (A549) were seeded in a 96-well optical-bottom plate. When the cells reached 70-80% confluency, the cells were treated with the compounds (DPUDs and DPTUD) dissolved in DMSO) (50 ∥M) for 12 h at 37° C. Following treatment, the cells were washed with PBS and fixed with 4% formaldehyde for 20 min at room temperature. Nucleus was stained with Hoechst diluted in PBS (1:10000). High-content microscopy and image analysis were performed to quantitate cell number (FIG. 3). All data are represented as mean±SD. The P-value was determined using one-way ANOVA with multiple comparisons w.r.t. DMSO. ns: P>0.05, *P<0.05, **P<0.01, ***P<0.001, ****P<0.0001.

### Example 4: Effect of Diphenyl Thiourea and Diphenyl Urea Derivatives in Blocking Cellular Entry and Infection of Influenza A Virus

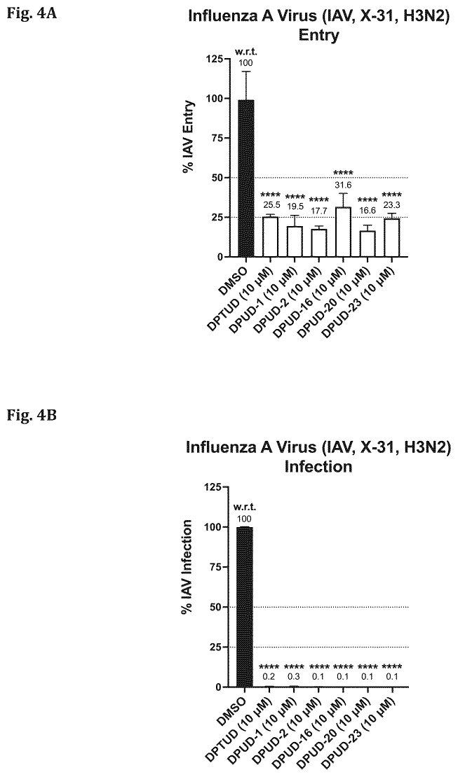

Human lung epithelial cells (A549) were seeded in a 96-well optical-bottom plate. When the cells reached 70-80% confluency, the cells were treated with the compounds, DPTUD and DPUDs (10 μM) dissolved in DMSO for 1 h at 37° C. Following pre-treatment, the cells were infected with influenza A virus (A/X-31, H3N2) strain in presence of the compounds DPTUDs and DPUD dissolved in DMSO (10 μM). The cells were fixed at 30 min and 10 h post-infection with 4% formaldehyde for 20 min at room temperature to check for virus endocytosis and infection, respectively. Virus endocytosis and infection were probed by indirect immunofluorescence against the viral HA and NP proteins, respectively. Nucleus was stained with Hoechst diluted in PBS (1:10000). High-content microscopy and image analysis were performed to quantitate virus endocytosis and infection (FIG. 4a,b). All data are represented as mean±SD. The P-value was determined using one-way ANOVA with multiple comparisons w.r.t. DMSO, ns: P>0.05, *P<0.05, **P<0.01, ***P<0.001, ****P<0.0001.

### Example 5: Effect of Diphenyl Thiourea and Diphenyl Urea Derivatives in Blocking Cellular Entry and Infection of SARS-CoV-2

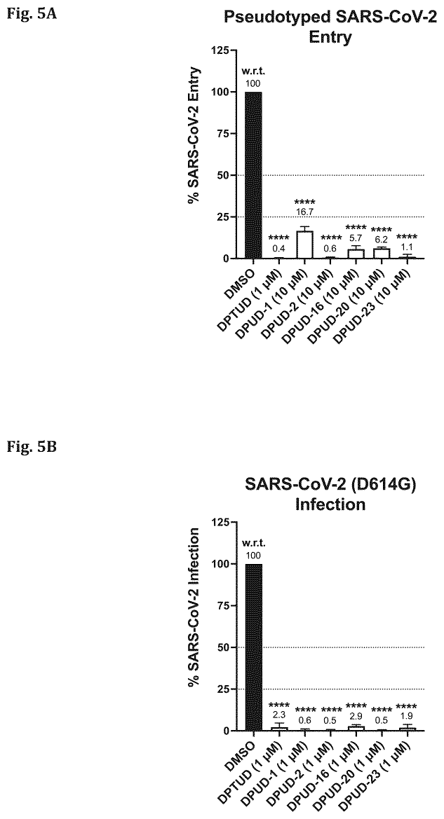

Viral entry inhibition assay was performed with pseudotyped SARS-CoV-2 in 293T-hACE-2 cells in presence of the compounds. SARS-CoV-2 infection was performed in Vero-E6 cells that were infected with SARS-CoV-2 (Alpha, B.1.1,7) strain at MOI: 0.2-0.6 in presence of the compounds (DPUDs and DPTUD) dissolved in DMSO (1 μM) for 24 h at 37° C. Following infection, the cells were washed with PBS and fixed with 4% formaldehyde for 20 min at room temperature. Indirect immunofluorescence was performed with anti-N (SARS-CoV-2) antibody to detect the infected cells expressing the N protein (FIG. 5a, b). Nucleus was stained with Hoechst diluted in PBS (1:10000). All data are represented as mean±SD. The P-value was determined using one-way ANOVA with multiple comparisons w.r.t. DMSO. ns: P>0.05, *P<0.05, **P<0.01, ***P<0.001, ****P<0.0001.

### Example 6. DPUDs Inhibit Influenza A Virus (IAV) and SARS-CoV-2 Infection in Tissue Culture Cells

Cells were infected with IAV or SARS-CoV-2 in presence of the compounds (10 μM), and the readout for infection was done by indirect immunofluorescence against IAV NP (10 h.p.i) or SARS-CoV-2 N (24 h.p.i.) Regorafenib (10 μM), sorafenib (10 μM), and bafilomycin A1 (BafA1) (50 nM) were used as positive controls in IAV infection. In SARS-CoV-2 infection, Favipiravir (10 μM), niclosamide (10 μM), and hydroxychloroquine (HCQ) (10 μM) were included as positive controls (FIG. 6a). Molecular chemical structures of DPU and the ‘hit’ compounds (DPUD-1, -2, -16, -20 and -23), which blocked IAV and SARS-CoV-2 infections by 95-100% (FIG. 6b). High-content microscopy images of IAV- and SARS-CoV-2-infected cells, treated with DMSO, DPU, DPU-1 and BafA1/niclosamide. Nuclei were stained with Hoechst (magenta), and the viral NP/N proteins (green) were detected by IIF (FIG. 6c). Scale bars, 50 μm. Reduced infection by DPUD-1, -2, -16, -20 and -23 in A549 cells infected with IAV Udorn (H3N2) and NYMC (H3N2) strains (FIG. 6d). e, Reduced infection by DPUD-1, -2, -16, -20 and -23 (10 μM) in Vero-E6 cells infected with SARS-COV 2 Delta (B.1.617.2) and Omicron (B.1.1.529) strains. n=3 biologically independent experiments (FIG. 6e). All data are represented as mean±SD. The P-value was determined using one-way ANOVA with multiple comparisons w.r.t. DMSO. ns: P>0.05, *P<0.05, **P<0.01, ***P<0.001, ****P<0.0001.

### Example 7: DPUD-1 Shows in Vivo Efficacy and High Barrier to Resistance Against Influenza A Virus (IAV) Infection

Six to ten week-old C57/BL6 mice (n=6/group) were infected with 1000 PFU IAV (WSN) on day 0. The infected mice were intraperitoneally administered DPUDs dissolved in DMSO/DMSO/Oseltamivir acid with 1 mg/kg of body weight twice daily from day 1 post-infection till day 6. Body weights of the mice were daily measured from day 0 till day 10, following which, the mice were sacrificed (FIG. 7a). Body weights of the uninfected mice and IAV-infected mice that were treated with DPUD-1, Oseltamivir acid, and DMSO were measured and represented in graph (FIG. 7b). The mice (n=5/group) were infected with 3000 PFU IAV (WSN) on day 0, following which they were intraperitoneally administered with DPUDs dissolved in DMSO/DMSO/Oseltamivir acid with 1 mg/kg of body weight twice daily for 3 days. RT-PCR of IAV (WSN) NP gene from the lungs of infected mice (n=3), sacrificed on day 6 post-infection (FIG. 7d). Virus titres from the lungs of infected mice (n=3), sacrificed on day 6 post-infection (FIG. 7e). A549 cells were infected with the virus (MOI=0.1) in presence of DPUD-1 dissolved in DMSO (10 μM) or oseltamivir acid (10 μM) or DMSO. Supernatants were collected after 24 h post-infection, and virus plaque assays were performed. (FIG. 7f) MDCK cells were infected with IAV (WSN) (MOI=0.1) in presence of DPUD-1 (1 μM) or oseltamivir acid (1 μM) or DMSO. Supernatants were collected every ⅔ days and new cells were infected with the viruses present in the supernatants. Serial passaging of the virus was carried out for 10 passages after which, virus titres for each compound treatment were determined (FIG. 7g). The P-value was determined using one-way ANOVA with multiple comparisons w.r.t. DMSO. ns: P>0.05, *P<0.05, **P<0.01, ***P<0.001, ****P<0.0001.

### Example 8: DPTUD and DPUDs Block Influenza A Virus (IAV) Endocytosis and Post-Entry Infection Events

IAV entry assays were performed in A549 cells with IAV (X-31). Cells were pre-treated with DPTUD and DPUDs dissolved in DMSO (10 μM) for 1 h, prior to virus addition, for each of the entry assays. In the binding assay, the viruses were allowed to bind to the receptors at 4° C. for 1 h in presence of DPTUD and DPUDs dissolved in DMSO (10 μM). All the IAV entry assays were performed in cells treated with 10 μM DPTUD and DPUDs dissolved in DMSO for respective durations of the assays: 30 min (endocytosis), 1 h (HA acidification), 2.5 h (uncoating), and vRNP nuclear import (4 h). Chlorpromazine (CPZ, 70 μM), an inhibitor of clathrin-mediated endocytosis, was included in the IAV endocytosis assay as the positive control, whereas bafilomycin A1 (BafA1) (50 nM), a vATPase inhibitor, was added as the positive control in the HA acidification, uncoating and vRNP nuclear import assays. After allowing virus entry for respective time points, indirect immunofluorescence (IIF) was performed for high-content microscopy. FIG. 8-b, Binding assay. FIG. 8-c, Endocytosis assay. FIG. 8-d, HA acidification assay. FIG. 8-e, Uncoating assay. FIG. 8-f, vRNP nuclear import assay. PM bypass infection assay. IAV (X-31) particles were allowed to bind to A549 cells at pH 6.8 at 4° C. for 1 h, following which, the cells were shifted to pH 5.0 at 37° C. for 2 min, inducing fusion at the PM. The cells were then shifted to pH 7.4 medium containing 20 mM NH₄Cl, and infection was allowed to happen at 37° C. for 10 h. After fixing the cells, IIF was performed to detect newly synthesized NP by high-content microscopy. (FIG. 8g) PM bypass infection assay in cells treated with DPUDs dissolved in DMSO (10 μM) till fusion. (FIG. 8h) PM bypass infection assay in cells in which, DPUDs dissolved in DMSO (10 μM) were added 1 h post-fusion and kept on for the rest 9 h. (FIG. 8i) Images from live cell microscopy, monitoring IAV entry. IAV (X-31) particles were labelled with SP-DiOC18(3) (green) and were allowed to enter A549 cells expressing Rab5-RFP (red) in presence of DMSO or DPUD-1 (10 μM). Post virus addition, images were acquired till 670 sec. Cell boundary is shown with dotted line. Scare bars, 20 μm. The P-value was determined using one-way ANOVA with multiple comparisons w.r.t. DMSO. ns: P>0.05, *P<0.05, **P<0.01, ***P<0.001, ****P<0.0001.

### Example 9: DPTUD and DPUDs Block General Endocytosis and Inhibit Post-Entry Events in IAV and SARS-CoV-2 Infections

A549 cells were pre-treated with DPTUD and DPUDs dissolved in DMSO (10 μM) and chlorpromazine (CPZ, 70 μM) for 1 h and 15 min, respectively. CPZ was included as a positive control as it blocks clathrin-mediated endocytosis. This was followed by addition of Alexa Fluor (AF) 488-conjugated EGF (30 ng/ml) and AF488-conjugated transferrin (30 μg/ml) to the cells. After allowing EGF and transferrin entry for 30 min and 10 min, respectively, the cells were fixed. AF488-conjugated CTXB (1 μg/ml) was allowed to enter cells for 15 min in presence of DPTUD and DPUDs dissolved in DMSO (10 μM) and methyl-β-cyclodextrin (MβCD, 10 mM), after pre-treatment with the compounds for 1 h. Since CTXB enters cells via clathrin-dependent as well as by caveolae- and clathrin-independent endocytosis, MβCD was included as a positive control, which selectively extracts cholesterol from the plasma membrane (PM), and thus, blocks clathrin/caveolin-dependent and -independent pathways (FIG. 1,2, FIG. 14). To probe vacuolar acidification, A549 cells pre-treated for 1 h with DPTUD and DPUDs dissolved in DMSO (10 μM) were incubated with LysoTracker Red DND-99 (1 μM) for 1 h in presence of the compounds. Bafilomycin A1 (BafA1) was included as a positive control as it blocks luminal acidification (FIG. 14). Confocal images of A549 cells treated with DPUD-1 dissolved in DMSO (10 μM) or DMSO for 1 h. Antibodies were used to stain EAA1 (cyan) and LAMP1 (red). Phalloidin-AF647 and Hoechst were used to stain actin filaments (yellow) and nuclei (magenta), respectively (FIG.-9a). Quantification of EAA1 fluorescence intensities in A549 cells treated with DPUDs (10 μM) for 1 h. EAA1 fluorescence intensities were measured from 100 cells for each compound (FIG.-9b). Time of DPUD addition assay for IAV (X-31) infection in A549 cells (FIG.-9c). Time of DPUD addition assay for SARS-CoV-2 (D614G) infection in Vero-E6 cells (FIG. 9d). Cells were either pre-treated with DPUDs dissolved in DMSO (10 μM and 1 μM for IAV and SARS-CoV-2 infections, respectively) for 2 h (−2 h) or DPUDs were added at different time points: 0 h, 2 h, 4 h, 6 h, and 8 h during infection. Cells were fixed 10 h post-infection (IAV) or 24 h post infection (SARS-CoV-2). Indirect immunofluorescence (IIF) was performed to detect IAV NP and SARS-CoV-2 N, followed by high-content imaging (FIG. 9c,d). Scale bars, 20 μm. The P-value was determined using one-way ANOVA with multiple comparisons w.r.t. DMSO. ns: P>0.05, *P<0.05, **P<0.01, ***P<0.001, ****P<0.0001.

### ADVANTAGES

DPTUD and DPUDs potently inhibit IAV and SARS-CoV-2 infections by primarily targeting their cellular entry and exhibit potent activity at a concentration 10 μM, which is seven-hundred times lesser than the concentration of known inhibitors of endocytosis such as chlorpromazine and methyl-β-cyclodextrin, respectively. Further the DPTUD and DPUDs did not show cytotoxicity even at 50 μM concentration (5× higher than working concentration) in contrast to high cytotoxicity (>75%) exhibited by the same concentration of known inhibitors of endocytosis. The compounds target post-entry events during virus infection, ensuring robust suppression of infection at multiple steps. Thus the DPTUD and DPUDs identified in this study are suitable for employment as antiviral compounds and administration of therapeutically effective amounts of these compounds would be a viable method of treating an infectious disease, a neurological disease, cancer, or a kidney disease

### REFERENCES

Krammer, F. et al. Influenza. Nat Rev Dis Primers 4, 3 (2018).
Nicola, M. et al. The socio-economic implications of the coronavirus pandemic (COVID-19): A review. Int J Surg 78, 185-193 (2020).
Cascella, M., Rajnik, M., Aleem, A., Dulebohn, S. C. & Di Napoli, R. Features, Evaluation, and Treatment of Coronavirus (COVID-19) in StatPearls (StatPearls Publishing, 2022).
Flerlage, T., Boyd, D. F. Meliopoulos, V., Thomas, P. G. & Schultz-Cherry, S. Influenza virus and SARS-CoV-2: pathogenesis and host responses in the respiratory tract. Nat Rev Microbiol 19, 425-441 (2021).
Tay, M. Z., Poh, C. M., Rénia, L., MacAry, P. A. & Ng, L. F P. The trinity of COVID-19: immunity, inflammation and intervention. Nat Rev Immunol 20, 363-374 (2020).
Short, K R., Kroeze, E. J. B. V, Fouchier, R. A. M. & Kuiken, T. Pathogenesis of influenza-induced acute respiratory distress syndrome. The Lancet Infectious Diseases 14, 57-69 (2014).
Schultz-Cherry, S. & Jones, J. C. Influenza Vaccines. in Advances in Virus Research vol. 77 63-84 (Elsevier, 2010).
Lou, F. et al. Understanding the Secret of SARS-CoV-2 Variants of Concern/Interest and Immune Escape. Front. Immunol. 12, 744242 (2021).
Kaufmann, S. H E., Dorhoi, Hotchkiss, R. S. & Bartenschlager, R. Host-directed therapies for bacterial and viral infections. Nat Rev Drug Discov 17, 35-56 (2018).
Kaufmann, S. H. K, Dorhoi, A., Hotchkiss, R. S. & Bartenschlager, R. Host-directed therapies for bacterial and viral infections. Nat Rev Drug Discov 17, 35-56 (2018).
Lesch, M. et al. RNAi-based small molecule repositioning reveals clinically approved urea-based kinase inhibitors as broadly active antivirals. PLoS Pathog 15, e1007601 (2019).
Banerjee, I., Yamauchi, Y., Helenius, A. & Horvath, P. High-Content Analysis of Sequential Events during the Early Phase of Influenza A Virus Infection. PLoS ONE 8, e68450 (2013).
Drożdżal, S. et al. An update on drugs with therapeutic potential for SARS-CoV-2 (COVID-19) treatment. Drug Resistance Updates 59, 100794 (2021).
de Vries, E. et al. Dissection of the influenza A virus endocytic routes reveals macropinocytosis as an alternative entry pathway. PLoS Pathog 7, e1001329 (2011).
Matlin, K. S., Reggio, H., Helenius, A. & Simons, K. Infectious entry pathway of influenza virus in a canine kidney cell line. J Cell Biol 91, 601-613 (1981).
Yamauchi, Y. & Greber, U. F. Principles of Virus Uncoating: Cues and the Snooker Ball. Traffic 17, 569-592 (2016).
Martin, K. & Helenius, A. Transport of incoming influenza virus nucleocapsids into the nucleus. J Virol 65, 232-244 (1991).
Banerjee, I. et al. Influenza A virus uses the aggresome processing machinery for host cell entry. Science 346, 473-477 (2014).
Schmidt, F. et al. Measuring SARS-CoV-2 neutralizing antibody activity using pseudotyped and chimeric viruses. J Exp Med 217, e20201181 (2020).
Sigismund, S., Lanzetti, L., Scita, G. & Di Fiore, P. P. Endocytosis in the context-dependent regulation of individual and collective cell properties. Nat Rev Mol Cell Biol 22, 625-643 (2021).
Ewers, H. & Helenius, A. Lipid-mediated endocytosis. Cold Spring Harb Perspect Biol 3, a004721 (2011).
Busschaert, N. et al. Squaramides as Potent Transmembrane Anion Transporters. Angew. Chem. Int. Ed. 51, 4426-4430 (2012).
Salam, R., Chowdhury, S. M., Marshall, S. R., Gneid, H. & Busschaert N. Increasing membrane permeability of carboxylic acid-containing drugs using synthetic transmembrane anion transporters. Chem. Commun. 57, 13122-13125 (2021).
Stauber, T. & Jentsch, T. J. Chloride in Vesicular Trafficking and Function. Annu. Rev. Physiol. 75, 453-477 (2013).

Marshall, S. R., Singh, A., Wagner, J. N. & Busschaert, N. Enhancing the selectivity of optical sensors using synthetic transmembrane ion transporters. Chem. Commun. 56, 14455-14458 (2020).
Chattopadhyay, K., Bhattacharyya, D. & Banerjee, K. K. Vibrio cholerae hemolysin. Implication of amphiphilicity and lipid-induced conformational change for its pore-forming activity. Eur J Biochem 269, 4351-4358 (2002).
Mondal, A. et al. Influenza virus recruits host protein kinase C to control assembly and activity of its replication machinery. eLife 6, e26910 (2017).
Edinger, T. O., Pohl, M. O. & Stertz, S. Entry of influenza A virus: host factors and antiviral targets. Journal of General Virology 95, 263-277 (2014).
O'Hanlon, R., Leyva-Grado, V. H., Sourisseau, M., Evans, M. J. & Shaw, M. L. An Influenza Virus Entry inhibitor Targets Class II PI3 Kinase and Synergizes with Oseltamivir. ACS Infect. Dis. 5, 1779-1793 (2019).
Kumar, N. et al. Host-Directed Antiviral Therapy. Clin Microbiol Rev 33, (2020).
Lin, B. & Waymouth, R. M. Urea Anions: Simple, Fast, and Selective Catalysts for Ring-Opening Polymerizations. J. Am. Chem. Soc. 139, 1645-1652 (2017).
Wang, M. et al. Effective approach to ureas through organocatalyzed one-pot process. Tetrahedron Letters 59, 1614-1618 (2018).
Bagdasarian, A. L. et al. Urea-Catalyzed Vinyl Carbocation Formation Enables Mild Functionalization of Unactivated C—H Bonds. Org. Lett. 22, 7775-7779 (2020).
Callison, J. et al. Origin of Impurities Formed in a Polyurethane Production Chain. Part 2: A Route to the Formation of Colored Impurities. Ind. Eng. Chem. Res. 51, 11021-11030 (2012).
Song, H-X. Han, Z.-Z. & Zhong C.-P. Concise and Additive-Free Click Reactions between Amines and CF3 SO3 CF3. Chemistry 25, 10907-10912 (2019).
Zhao, Y. et al. Hydrosilane-Assisted Synthesis of Urea Derivatives from CO 2 and Amines, J. Org. Chem. 85, 13347-13353 (2020).
Zhou, S., Yao, T., Yi, J., Li, D. & Xiong, J. A Simple and Efficient Synthesis of Diary 1 Ureas with Reduction of the Intermediate isocyanate by Triethylamine. Journal of Chemical Research 37, 315-319 (2013).
Park, J. H., Yoon, J. C. & Chung, Y. K. Cobalt/Rhodium Heterobimetallic Nanoparticle-Catalyzed Oxidative Carbonylation of Amines in the Presence of Carbon Monoxide and Molecular Oxygen to Ureas. Adv. Synth. Catal. 351, 1233-1237 (2009).
Ricci, A. et al. Effect of Cl-substitution on rooting- or cytokinin-like activity of diphenylurea derivatives. J Plant Growth Regul 23, 261-268 (2004).
Daly, J. L. et al. Neuropilin-1 is a host factor for SARS-CoV-2 infection. Science 370, 861-865 (2020).

1. A method of inhibiting endocytosis in a cell comprising treating the cell with a compound of Formula-I or a pharmaceutical composition thereof, wherein the compound of Formula-I is: 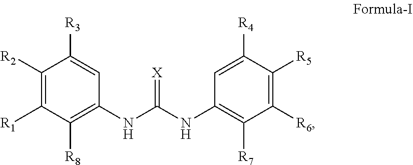
wherein
X is S or O;
R₁ is H, halogen, alkoxy, or haloalkyl;
R₂ is H, halogen, haloalkyl, haloalkoxyl, alkoxyl, alkyl, nitro, or benzyl;
R₃ is H, halogen, alkyl, or haloalkyl;
R₄ is H, halogen, alkyl, or haloalkyl;
R₅ is H, haloalkyl, haloalkoxyl, halogen, alkoxyl, alkyl, nitro, or benzyl;
R₆ is H, halogen, alkoxy, or haloalkyl;
R₇ is H, haloalkyl, alkyl, or halogen; and
R₈ is H, haloalkyl, alkyl, or halogen.

2. The method of claim 1, wherein the cells are treated with the compound of Formula-I at a concentration of 0.1 μM to 25 μM for 10 minutes to 24 hours at a temperature of 37° C.

3. The method of claim 1, wherein the compound of Formula-I inhibits the endocytosis of endocytic cargoes comprising at least one material selected from the group consisting of Transferrin, EGF, and cholera toxin, in the cell.

4. The method of claim 1, wherein the compound of Formula-I inhibits the endocytosis of pathogens comprising viruses, bacteria and fungi into the cell.

5. The method of claim 4, wherein the virus is selected from the group consisting of SARS-CoV-2 and Influenza.

6. A method of treating an infectious disease, a neurological disease, cancer, or a kidney disease in a subject in need thereof, comprising administering to the subject a therapeutically effective amount of a compound of Formula-I or a pharmaceutical composition thereof, wherein the compound of Formula-I is: 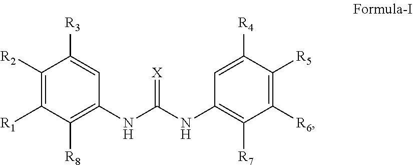
wherein
X is S or O;
R₁ is H, halogen, alkoxy, or haloalkyl;
R₂ is H, halogen, haloalkyl, haloalkoxyl, alkoxyl, alkyl, nitro, or benzyl;
R₃ is H, halogen, alkyl, or haloalkyl;
R₄ is H, halogen, alkyl, or haloalkyl;
R₅ is H, haloalkyl, haloalkoxyl, halogen, alkoxyl, alkyl, nitro, or benzyl;
R₆ is H, halogen, alkoxy, or haloalkyl;
R₇ is H, haloalkyl, alkyl, or halogen; and
R₈ is H, haloalkyl, alkyl, or halogen.

7. The method of claim 6, wherein the infectious diseases comprise diseases caused by viral, bacterial, and fungal infections.

8. The method of claim 6, wherein the infectious diseases comprise diseases caused by Influenza A Virus and Severe acute respiratory syndrome coronavirus 2.

9. The method of claim 6, wherein the therapeutically effective amount of the compound of Formula-I ranges from 1 mg to 10 mg/kg of body weight of the subject.

10. The method of claim 6, wherein the compound is administered in a form of the pharmaceutical composition.

11. The method of claim 6, wherein the compound is administered orally, intraperitoneally, intravenously, or intranasally.

12. A compound of Formula-I, 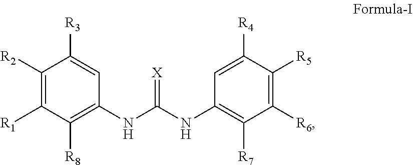
wherein
X is S or O;
R₁ is H, halogen, alkoxy, or haloalkyl;
R₂ is H, halogen, haloalkyl, haloalkoxyl, alkoxyl, alkyl, nitro, or benzyl;
R₃ is H, halogen, alkyl, or haloalkyl;
R₄ is H, halogen, alkyl, or haloalkyl;
R₅ is H, haloalkyl, haloalkoxyl, halogen, alkoxyl, alkyl, nitro, or benzyl;
R₆ is H, halogen, alkoxy, or haloalkyl;
R₇ is H, haloalkyl, alkyl, or halogen; and
R₈ is H, haloalkyl, alkyl, or halogen.

13. The compound of Formula-I, as claimed in claim 12, wherein X is S; R₂, R₅, R₇, and R₈ are H; and R₁, R₃, R₄, and R₆ are haloalkyl.

14. The compound of Formula-I, as claimed in claim 12, wherein X is O; ═R₂, R₅, R₇, and R₈ are H; and R₁, R₃, R₄, and R₆ are haloalkyl.

15-18. (canceled)

19. The method of claim 1, wherein in the compound of Formula-I, X is S; R₂, R₅, R₇, and R₈ are H; and R₁, R₃, R₄, and R₆ are haloalkyl.

20. The method of claim 1, wherein in the compound of Formula-I, X is O; R₂, R₅, R₇, and R₈ are H; and R₁, R₃, R₄, and R₆ are haloalkyl.

21. The method of claim 1, wherein in the compound of Formula-I, X is O; R₂ and R₅ are haloalkyl; and R₁, R₃, R₄, R₆, R₇, and R₈ are H.

22. The method of claim 1, wherein in the compound of Formula-I, X is O; R₂, R₅, R₇, and R₈ are H; and R₁, R₃, R₄, and R₆ are Halogen.

23. The method of claim 1, wherein in the compound of Formula-I, X is O; R₂, R₅, R₇, and R₈ are H; and R₁, R₃, R₄, and R₆ are each independently F or Br.

24. The method of claim 1, wherein in the compound of Formula-I, X is O; R₂ and R₅ are haloalkoxyl; and R₁, R₃, R₄, R₆, R₇, and R₈ are H.
